SEQUENCE LISTING (11 sequences)

SEQ ID NO: 1 - DNA, 62 bp, synthetic construct
  source 1..62: mol_type = other DNA
atgaaagagaagatgttcaaaagaaaacattcacaaaatgggtaaatgcacaattttctaag

SEQ ID NO: 2 - RNA, 30 nt, synthetic construct
  source 1..30: mol_type = other RNA
tttacccattttgtgaatgttttcttttga

SEQ ID NO: 3 - RNA, 14 nt, synthetic construct
  source 1..14: mol_type = other RNA
gccctgaagagggc

SEQ ID NO: 4 - DNA, 257 bp, synthetic construct
  source 1..257: mol_type = other DNA
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgca

SEQ ID NO: 5 - RNA, 11 nt, synthetic construct
  source 1..11: mol_type = other RNA
aatttttggag

SEQ ID NO: 6 - RNA, 31 nt, synthetic construct
  source 1..31: mol_type = other RNA
caggttttctgacttcggtcggaaaacccct

SEQ ID NO: 7 - RNA, 107 nt, synthetic construct
  source 1..107: mol_type = other RNA
cccaatttcactggtctacaatgaaagcaaaacagttctcttccccgctccccggtgtgtgagaggggctttgatccttctctggtttcctaggaaacgcgtatgtg

SEQ ID NO: 8 - RNA, 149 nt, synthetic construct
  source 1..149: mol_type = other RNA
aatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 9 - DNA, 484 bp, synthetic construct
  source 1..484: mol_type = other DNA
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaagagggccctgaagagggcccttttcttatgatagggacttagggtgtttacccattttgtgaatgttttcttttgaaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 10 - DNA, 9227 bp, synthetic construct
  source 1..9227: mol_type = other DNA
agcgcgcaaagccactactgccacttttggagactgtgtacgtcgagggcctgcagtaacagctaggacgtccgcatgcggtaggttaccttttcccccccaacctaaagcgaacaacgtatccaaccagagtttgaggaccggagttcactaagaggacggagacggagggtttcacgactctaatgtccacactccgtggtacggtccagagaatgacaaacattaatttatgtatgtgtaaaacacacaaacacacgtggaaatatttcagtttccactatcattgggtaaattcaaggatgagttaaaatgaaaggtccctattgattgatgaaaaagaaaaactctacctcagagcgacacatcgggtccgacctcacgtcaccgtggtagagccgagtgacgttcgaggaggagggaccaagtgcgataagaggacggagtcggaggggttgttgatcctgatgtccgagtggagcggtatggaccgattaaaaaacataaaaatcatctctgtcccaaagtgacacaatcggtcctaccagagctagaggactggaacactaggcggacggagacggagggtttcacgaccctaatgtccgtactcgttggagtgggtcgaccctattgatgaaaaatgtccaactataagaaaacctgaaaaggggacacatttttatatgatataaacaatacatgtataatacatgtatgtctgtgtttaacctggtaagagtcatattactaagagtccaaaaaaaaaaaaaaaaactccaccccttgatctattaatacctgtagaaaggtatgatcgtatagttatagatggagtaagaaaaattataaaaacgatcataaggtaacatacttacaggatactaaatgaattggacaggtagttataaacaaaggtccaaaaacgataatattacgacgacgtttcatgtaggagtgtgtagaaataaaacagataagtataaagacattctatccaatgatttcaaccttgacggtttaattgtgatagtatgataaaacaaaaaattaaaattaaaaaattttttacattttacacgttaaagttctcctctttgaacttgtgttcctcgttttagataaaaatattgtaggataattttcgaacgaaatgtatttctaaaactttcttatcgtatttatgttctaaagataaaattaacctaagaatcccgattattttattagtcggaatcgtgaataaataaataaaaaaaactctccctcagagcgagacaacaggtacgacctcacgtcaccgcactagagccgagtgacgttcgaggtggagtactcaagtgtggtaagaggacggagtcagagggctcatcgaccctgaggtccgcgggagatgtttcgggcagattaaaaaaaacataaaaatcatctctgtcccaaagtgacacaatcggtcctaccagaactagaggactggaacactagacgggcggagccggagggtttcacgaccctaatatccgaactcggtgacgagggccggtcgtgaataaaaatattaagaagtactaatgacacaatgacagggtacccggcggtcccggtcgatccaaccggtgagggagagacgcgcgagcgagcgagtgactccggcccgctggtttccagcgggctgcgggcccgaaacgggcccgccggagtcactcgctcgctcgcgcgtctctccctcaccggttgaggtagtgatccccaaggaggatcgtgcgcatcttagtcttttagactcttcttttgaatttttggaaacttagattggatgagtttatttgaaacttatataaataactttataaattaaacaaataaaaaataaaaaaataaaatctctgttccagagtgatttcacgtccgatctcacgtcactgtgttagtaccgataccgagtgacgttggagtttgaggacccgagttcgctaagacaacggaatccaagcggtcctcgaccgtgatgtccacggtggtgtggaccgaaaaaacaaaacaaaaaaaacccatctcttcccctaaaccatacaacgaatccgactagaacttgatcggagtgcgttaggacgaagccgaaaggttttacaaccctaatatccgtactcggtacgcggactggaactaatggagctactacacatacagtatgtaacctcccgtttctgtagagacttaaggagtgttgtcggtaccgtcccccgtccatgggtgtatgcgcaaaggatcctttggtctcttcctagtttcggggagagtgtgtggcccctcgccccttctcttgacaaaacgaaagtaacatctggtcactttaaccctccccaaaaggctggcttcagtcttttggacgaggtttttaaagttttcttttgtaagtgttttacccatttgtgggattcagggatagtattcttttcccgggagaagtcccgggagaaacgccttcacgcagacatcgctcggtcccttcctgtagttgaggtgaaagctactcccactctagttccacggtaaaggtgtggggaggtgactatacacttagtgtttcgtgtcaaggaataagccaagctatttgttataagattttctgataattttggcgagcaaagaactcaaacactggcgaacatttccgatacgtttactcagtcacgactaaccgacttttgtcggttagtgtcgaggatacaacaatctaggtggtccctgtcccaataaaaatctcgctcgaagaggaggtaccacatgtctcggattctgggtccgtggccccacccccactccgagtccgtcgtccacaacccccccccccccgtcggtgtacagacccaattaatattggtccgtagagcccacaggggtccggaacggaggaatgtacccgtcggatctgggcatcaccccatgggtacaatgtctactgtctgactacgtatctcttcaattcagcacccctcaaatgaaagaggatttaacaggacaatgatctacttaaacaaaaacaaagtaaaacaaaacaaaaactctgtctcagagtacaacagtgggtctgacctcacgtcaccgaggtagagccgagtgatatcggaggcggaggactcaagttcactaagaggacggagtcggagggttcatcgacccacgtacggtggtgcggaccgattaaaaacataaaaatcatatctaccccaaagtggtacaaccggtctgaccagagcttgaggactggagttcactaggtggggcgcgaatcggagggtttcacgaccctaatatccgcactcggtggtgtggtccggtacttaaacaaaagttataaataaataaaacataaaagataaaaactctacctcagagcgagacgacaggtccgatctcacgtcaccacactagaaccgagtgacatcggaggtggaggactcaagttcactaagaggacggaatcggagggctcatcgaccctaatgtccgcgggtggtagtgtgggccgattaaaaacataaaaatcatctctaccccaaagtggtacaaccggtgcgaccagagtttgactggagttaactaggtgggtggaactggagggtttcacgaccctaatgtctggactcggtgtcgcgggtcgggaagttataaataaatttaaacggacgaccgattgaagagtaacgtggacccgagatcacattaatttaatgaagtaagagaaaaattttgaaaaatgaaaaaagaaaaaacacaaaaagtaagagaatagatgctctcggtgttatgaacttctgtggttaactatggggaatcagtgtagactcgatttgtgaaagtcaaggatgtcgacaaagaattagaatccagtgtaccaaagaagggtacgacaagaagggtctgtcgtaaaaaaaaaaaaaactctcagagtgagacaacgggtccgacctcatgtcatcgtgttagagtcaaatgacgttggagacggaaggtccaagtccactaagaggacgaagtcggaggactcatcgaccctgatgtcctcgcacggtggtgcgggccgattaaaaacataaaaatcatctctgtcccaaagtggtacaaccggtccgaccagagcttgaggaatggaacactaggcggacagagccggagggtttcaccaccctaatgtccacactcggtggtgcggaccaagaatgtaaataaaaccttatttaaatctatgtgtcttttcaacgtttctattctcaaaggtatattgggagtgggtcaaacggaagggattacaattgtagaatgtaatagtaccatgtaaacagttttgattctgtgaaaaaaaagattatttttattatctctactccagagtgatataacaggtccgaccagagtttgaaactcgagttcgtcaggagggtggaggtggagggtttcacgaccctaatgtccgtacttggtggtgtgggtcggatgtaacaatacaatgatgagaggtctgataagtctaaagtggttaaaaagataggtatcggaaaaaaagacaaagtcctaggggtgatgcccagatccgacgggtacattcctccgttccggacccctgtgggctctacggaccaatattaattgggtctgtacaccgacgggggggggggggttgtggacgacggactcggagtgggggtggggccacggacccagaatccgagacatgtggtacctcctcttcgagcgagatttttattgggacagggaccacctagattgttgtatcctcgacactaaccgacaaaagtcggttagtcgtgactgagtaaacgtatcggaaatgttcgccagtgtttgagttctttgctcgccaaaattatcagaaaatcttataacaaatagcttggcttattccttgacacgaaacactaagtgtatagtcacctccccacacctttaccgtggaactagagtgggagtagctttcacctcaactacaggaagggaccgagcgatgtctgcgtgaaggcgtttctcccgggacttctcccgggaaaagaatactatccctgaatcccacaaatgggtaaaacacttacaaaagaaaactttaaaaacctcgtccaaaagactgaagccagccttttggggagggttaaagtgaccagatgttactttcgttttgtcaagagaaggggcgaggggccacacactctccccgaaactaggaagagaccaaaggatcctttgcgcatacaccctaggttaagtcctgtggtatggtgacataccaattaaaatttttgagtttacaaccctcaaacgaaatagtggcgacatagaatagtagagcaatctattaaaacatagacctcggacgagagatcgtacttattcatttttacacctgaaacactaaatgtctagactataaatacaaaaacacaaaaatgaaattcatgatctgtttcatttagattctccagacaccatgacgtgatctcttaacaatgaactaataccacccggttcaccaacttttaaatggattgttatgatattgtatccggtataaagtattaaaattagtgttccgttaacacttttaaaatagtttacaaattattttcgttccacttcttccactatcgaaatttaaatgaacggtaaaaacccgtgtgttttcattaaacgagacggtgaatctcaatattccagtttcaccctcatttattagaaacataatccttacgccggcgagatcctccttggggatcactacctcaaccggtgagggagagacgcgcgagcgagcgagtgactccggcgggcccgtttcgggcccgcagcccgctggaaaccagcgggccggagtcactcgctcgctcgcgcgtctctccctcaccggttggatctccggcggtcccggtataaagagttaaaaatttaaaaagtttttttaattaggaattacacgtataaaaacttaacaattatattgaaaaactccactacagaagtacacaaagttgatgaatttttgaaaatttgtcatatattattttttagaaggtccggtgagtgtggacattagggtcgtgaaaccctccgactccacccgtctagtggactcccgtcctcaagctctggtcggaccggttatatatatataagtatataagtatatatatatatataagtatataagtatatatatataagtatataagtatatatatatatatatatatatatcgttttggagtagagattattttatgtttttaatcgactcgcaccactacctacggacatcagggtcgatgagccctccgactccgtcctcttagagaacttggaccctccacctccaacgtcactcgactctaccacggtgacgggaggtcggactcactgtctcgctctgagccagaggtttttttttgttgtttttttagaaggtaggaacagagggtaggtggggaaggggggtcgtacatgaacgtctgaaatacgtatatgtcactcatgacatatatgtgtttattatttttttagtatatatattatatacattaaggggaaatgtactttccatcgtgtgaccagacatgtcagacagacgtgacacgataaagtgaaatataaaaatatcaaactgtctcaagattgtaaagaaaaaaaaaaaaaaattgtctcagaacaaggactaacaatttaaaatttcgtaggatttcaaaccaaagtgtgaacttacttatggtacattcctaagtgaatgtatctacaccaacggacttagaattcttattttattgtaacaaacataaataaatttaatcacaaggaaaataccaaacggactttcgtgttgttttaggagtggttctataatgttaatactgagggtatgtccatttgacaaatctctaaccgttcgtggaaaattactttcctcagtcggtcgaatcacacgtcataaataaagacggccttctccctcgaagtccctgtctgaaaccaaatcagtacttcggaggtcgtgagggttcgccaacaccaactggttcgttaaatacgaaaatggaaagatgaaggtctccgaacaaatgaatagtcattcgtaattaaatcacaggggagtctacggaaaatgaaagaagaaaagacggatcttattcgacgagaaggttaaaacgtcgatgtacaaaggtggggtcaaccttaaagaggtattgtaggtaacatcgataggaagttagatgtcggagataaaggacaatatcgaccagtccagattagggagttttatgagacaggggacgaagggaatagacgaccggtggaaaaagggggtgtatgtgtgacggtacagggtgggaagtgagttcaacaagggacggtggagttgtttaaattcaggtattttggtaggttacccgagctcgggacgtcctagtaacagtgtacactcgttttccggtcgttttccggtccttggcatttttccggcgcaacgaccgcaaaaaggtatccgaggcggggggactgctcgtagtgtttttagctgcgagttcagtctccaccgctttgggctgtcctgatatttctatggtccgcaaagggggaccttcgagggagcacgcgagaggacaaggctgggacggcgaatggcctatggacaggcggaaagagggaagcccttcgcaccgcgaaagagtatcgagtgcgacatccatagagtcaagccacatccagcaagcgaggttcgacccgacacacgtgcttggggggcaagtcgggctggcgacgcggaataggccattgatagcagaactcaggttgggccattctgtgctgaatagcggtgaccgtcgtcggtgaccattgtcctaatcgtctcgctccatacatccgccacgatgtctcaagaacttcaccaccggattgatgccgatgtgatcttcttgtcataaaccatagacgcgagacgacttcggtcaatggaagcctttttctcaaccatcgagaactaggccgtttgtttggtggcgaccatcgccaccaaaaaaacaaacgttcgtcgtctaatgcgcgtctttttttcctagagttcttctaggaaactagaaaagatgccccagactgcgagtcaccttgcttttgagtgcaattccctaaaaccagtactctaatagtttttcctagaagtggatctaggaaaatttaatttttacttcaaaatttagttcgggttagacttattacaatgttggttaattggttaagactaatctttttgagtagctcgtagtttactttgacgttaaataagtatagtcctaatagttatggtataaaaactttttcggcaaagacattacttcctcttttgagtggctccgtcaaggtatcctaccgttctaggaccatagccagacgctaaggctgagcaggttgtagttatgttggataattaaaggggagcagtttttattccaatagttcactctttagtggtactcactgctgacttaggccactcttaccgttttcaaatacgtaaagaaaggtctgaacaagttgtccggtcggtaatgcgagcagtagttttagtgagcgtagttggtttggcaataagtaagcactaacgcggactcgctctgctttatgcgctagcgacaattttcctgttaatgtttgtccttagcttacgttggccgcgtccttgtgacggtcgcgtagttgttataaaagtggacttagtcctataagaagattatggaccttacgacaaaaaggcccctagcgtcaccactcattggtacgtagtagtcctcatgcctattttacgaactaccagccttctccgtatttaaggcagtcggtcaaatcagactggtagagtagacattgtagtaaccgttgcgatggaaacggtacaaagtctttgttgagaccgcgtagcccgaagggtatgttcgctatctaacagcgtggactaacgggctgtaatagcgctcgggtaaatatgggtatatttagtcgtaggtacaaccttaaattagcgccggagctgcaaagggcaacttataccgagtattgtggggaacataatgacaaatacattcgtctgtcaaaataacaagtactactatataaaaatagaacacgttacattgtagtctctaaaactctgtgcccggtctcgacgt

SEQ ID NO: 11 - DNA, 207 bp, synthetic construct
  source 1..207: mol_type = other DNA
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatc

(19) United States
(12) Patent Application Publication
(10) Pub. No.: US 2025/0215426 A1
(43) Pub. Date: Jul. 3, 2025

(54) COMPOSITIONS AND METHODS FOR THE TREATMENT OF MUSCULAR DYSTROPHIES

(71) Applicant: ASTELLAS GENE THERAPIES, INC., South San Francisco, CA (US)

(72) Inventors: Yukari MORIYA, Tsukuba (JP); Katsuro YOSHIOKA, Tsukuba (JP); Eiji YOSHIMI, Tsukuba (JP); Dwaipayan SEN, San Francisco, CA (US); Fabrizia URBINATI, Foster City, CA (US); Takatomo ARAI, Tsukuba (JP); Mariko MANDA, Tsukuba (JP); Daisuke SATO, Tsukuba (JP)

(21) Appl. No.: 18/853,514

(22) PCT Filed: Apr. 5, 2023
(86) PCT No.: PCT/US2023/065377

§ 371 Date: Oct. 2, 2024

Related U.S. Application Data
(60) Provisional application No. 63/327,532, filed on Apr. 5, 2022.

(51) Int. Cl. C12N 15/113; A61K 35/76; C12N 15/86

(52) U.S. Cl. CPC C12N 15/113; A61K 35/76; C12N 15/86; C12N 2310/11; C12N 2310/3519; C12N 2320/33; C12N 2320/34; C12N 2750/14143

(57) ABSTRACT

Some embodiments relate to recombinant adeno-associated virus (rAAV) delivery of polynucleotides for treating muscular dystrophy resulting from the duplication of DMD exon 2. The invention provides rAAV products and methods of using the rAAV in the treatment of muscular dystrophy

### CROSS-REFERENCE TO RELATED APPLICATIONS

This application is a national phase filing under 35 C.F.R. § 371 of and claims priority to PCT Patent Application No. PCT/EP2023/065377, filed on Apr. 5, 2023, which claims the priority benefit under 35 U.S.C. § 119 of U.S. Provisional Patent Application No. 63/327,532, filed on Apr. 5, 2022, the contents of which are hereby incorporated in their entireties by reference.

### CROSS REFERENCE TO RELATED APPLICATION(S)

This application claims the benefit of priority to U.S. Provisional Application Ser. No. 63/327,532, which was filed Apr. 5, 2022, the entire disclosure of which is hereby incorporated by reference as if set forth in its entirety.

### SEQUENCE LISTING

The instant application contains a Sequence Listing which has been submitted electronically in XML format and is hereby incorporated by reference in its entirety. Said XML copy, created on Apr. 3, 2023, is named “51037-062WO2_Sequence_Listing_4_3_23” and is 20,956 bytes in size.

### FIELD OF THE DISCLOSURE

The presently disclosed subject matter relates to the field of therapeutic treatment of muscular dystrophies in patients, such as Duchenne muscular dystrophy in human patients.

### BACKGROUND OF THE DISCLOSURE

Duchenne muscular dystrophy (DMD) is a severe X-linked muscle degenerative disease caused by the absence of the cytoskeletal protein dystrophin. In contrast, mutations that preserve a reading frame of the gene that encodes dystrophin, resulting in a partially functional protein, elicit the milder Becker muscular dystrophy (BMD). The dystrophin protein provides stability to the sarcolemma (i.e., the cell membrane of muscle cells) by linking the intracellular cytoskeletal network to the extracellular matrix. In the absence of dystrophin, muscle contraction mechanically stresses the cell membrane, inducing progressive damage to the myofibers. Initially, skeletal muscles are predominantly affected; however, as the disease progresses, the damage extends to cardiac muscle and death is caused by respiratory or cardiac failure. DMD is one of the most common genetic diseases, affecting an estimated 1 out of every 3,600 male births each year. DMD is a debilitating disease that progressively worsens over the short (approximately 25 year) lifespan of those affected. Recently, gene therapy approaches involving the delivery of interfering ribonucleic acids for the targeted skipping of exon 2 of the dystrophin gene have been developed. However, there is a need in the art for improved efficacy of gene therapy approaches to skip DMD exon 2 for patients having DMD or BMD.

### SUMMARY OF THE DISCLOSURE

The present disclosure provides compositions and methods for preventing disease, delaying the progression of disease, and/or treating patients with one or more 5′ mutations of the dystrophin (DMD) gene. The compositions and methods are based on the identification of an efficacious approach to skip translation of DMD exon 2, the method including the step of administration of a U7 small nuclear ribonucleic acid (snRNA)-directed antisense polynucleotide to the exonic splicing enhancer (ESE) of exon 2, the skipping of which can generate a functional N-terminally truncated dystrophin isoform.

The disclosure contemplates methods of ameliorating Duchenne muscular dystrophy (DMD) or Becker muscular dystrophy (BMD) in a patient with a 5′ mutation in the DMD gene including the step of administering an antisense polynucleotide (e.g., an interfering ribonucleic acid (RNA)) to the patient, wherein the patient has a DMD exon 2 duplication and/or the patient has a frameshift mutation in any one of exons 1-4 of an endogenous DMD gene.

In one aspect, the disclosure provides a transgene encoding a RNA molecule, wherein the RNA molecule includes: (i) a BoxB RNA element; and (ii) an antisense polynucleotide of from 10 to 100 (e.g., 11 to 99, 12, to 98, 13 to 97, 14, to 96, 15 to 95, 20 to 90, 30 to 80, 40 to 70, or 50 to 60) nucleotides in length having complementarity sufficient to hybridize to a region within a protein-encoding mRNA transcript (e.g., exon 2 of a human dystrophin RNA transcript).

In some embodiments, the BoxB RNA element has a nucleic acid sequence that is at least 85% (e.g., at least 86%, 87%, 88%, 89%, 90%, 91%, 92%, 93%, 94%, 95%, 96%, 97%, 98%, 99%, or 99.9%) identical to the nucleic acid sequence of SEQ ID NO: 3. In some embodiments, the BoxB RNA element has a nucleic acid sequence that is at least 90% identical to the nucleic acid sequence of SEQ ID NO: 3. In some embodiments, the BoxB RNA element has a nucleic acid sequence that is at least 95% identical to the nucleic acid sequence of SEQ ID NO: 3. In some embodiments, the BoxB RNA element has a nucleic acid sequence that is at least 96%, 97%, 98%, or 99% identical to the nucleic acid sequence of SEQ ID NO: 3. In some embodiments, the BoxB RNA element has the nucleic acid sequence of SEQ ID NO: 3.

In some embodiments, the BoxB RNA element is positioned 5′ to the antisense polynucleotide or the BoxB RNA element is positioned 3′ to the antisense polynucleotide. In some embodiments, the BoxB RNA element is positioned 5′ to the antisense polynucleotide.

In some embodiments, the mRNA transcript is a human dystrophin mRNA transcript, a human fukutin mRNA transcript, a human gamma-sarcoglycan mRNA transcript, a human dysferlin mRNA transcript, a human myotonic dystrophy protein kinase mRNA transcript, a human laminin subunit alpha 2 mRNA transcript, a human usherin mRNA transcript, a human collagen alpha-1(VII) chain mRNA transcript, or a human activin A receptor type 1 mRNA transcript. In some embodiments, the mRNA transcript is a human dystrophin mRNA transcript.

In some embodiments, the antisense polynucleotide includes a portion of from 25 to 40 (e.g., 25 to 40, 26 to 40, 27 to 40, 28 to 40, 29 to 40, 30 to 40, 31 to 40, 32 to 40, 33 to 40, 34 to 40, 35 to 40, 36 to 40, 37 to 40, 38 to 40, or 39 to 40) nucleotides in length having complementarity sufficient to hybridize over the length of, or within, a region of exon 2 of a human dystrophin RNA transcript, wherein the region begins at residue 17 of SEQ ID NO: 1 and ends at residue 46 of SEQ ID NO: 1. In some embodiments, the portion of the antisense polynucleotide is from 25 to 35 nucleotides in length. In some embodiments, the portion of the antisense polynucleotide is from 26 to 34 nucleotides in length. In some embodiments, the portion of the antisense polynucleotide is from 27 to 33 nucleotides in length. In some embodiments, the portion of the antisense polynucleotide is from 28 to 32 nucleotides in length. In some embodiments, the portion of the antisense polynucleotide is from 29 to 31 nucleotides in length. In some embodiments, the portion of the antisense polynucleotide is 30 nucleotides in length. In another example, in some embodiments, the portion of the antisense polynucleotide is from 31 to 39 nucleotides in length. In some embodiments, the portion of the antisense polynucleotide is from 32 to 38 nucleotides in length. In some embodiments, the portion of the antisense polynucleotide is from 33 to 37 nucleotides in length. In some embodiments, the portion of the antisense polynucleotide is from 34 to 36 nucleotides in length. In some embodiments, the portion of the antisense polynucleotide is 30 nucleotides in length.

In some embodiments, the antisense polynucleotide does not have complementarity to exon 2 of a human dystrophin RNA transcript at a site located 5′ to residue 17 of SEQ ID NO: 1. In some embodiments, the antisense polynucleotide does not have complementarity to exon 2 of a human dystrophin RNA transcript at a site located 3′ to residue 46 of SEQ ID NO: 1.

In some embodiments, the antisense polynucleotide has a nucleic acid sequence that is at least 70% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9%) complementary to the region beginning at residue 17 of SEQ ID NO: 1 and ending at residue 46 of SEQ ID NO: 1. In some embodiments, the antisense polynucleotide has a nucleic acid sequence that is at least 75% complementary to the region beginning at residue 17 of SEQ ID NO: 1 and ending at residue 46 of SEQ ID NO: 1. In some embodiments, the antisense polynucleotide has a nucleic acid sequence that is at least 80% complementary to the region beginning at residue 17 of SEQ ID NO: 1 and ending at residue of SEQ ID NO: 1. In some embodiments, the antisense polynucleotide has a nucleic acid sequence that is at least 85% complementary to the region beginning at residue 17 of SEQ ID NO: 1 and ending at residue 46 of SEQ ID NO: 1. In some embodiments, the antisense polynucleotide has a nucleic acid sequence that is at least 90% complementary to the region beginning at residue 17 of SEQ ID NO: 1 and ending at residue 46 of SEQ ID NO: 1. In some embodiments, the antisense polynucleotide has a nucleic acid sequence that is at least 95% complementary to the region beginning at residue 17 of SEQ ID NO: 1 and ending at residue 46 of SEQ ID NO: 1. In some embodiments, the antisense polynucleotide has a nucleic acid sequence that is at least 96%, 97%, 98%, or 99% complementary to the region beginning at residue 17 of SEQ ID NO: 1 and ending at residue 46 of SEQ ID NO: 1. In some embodiments, the antisense polynucleotide has a nucleic acid sequence that is fully complementary to the region beginning at residue 17 of SEQ ID NO: 1 and ending at residue of SEQ ID NO: 1.

In some embodiments, the antisense polynucleotide includes at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes from 10 to 30 (e.g., 10, 11, 12, 13, 14, 15, 16, 17, 18, 19, 20, 21, 22, 23, 24, 25, 26, 27, 28, 29, or 30) contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes from 12 to 30 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes from 15 to 30 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes from 18 to 30 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes from 21 to 30 contiguous nucleotides that are fully complementary to a contiguous polynucleotide segment of equal length within the region beginning at residue of SEQ ID NO: 1 and ending at residue 46 of SEQ ID NO: 1. In some embodiments, the antisense polynucleotide includes from 24 to 30 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes 30 contiguous nucleotides that are fully complementary to a contiguous polynucleotide segment of equal length within the region beginning at residue 17 of SEQ ID NO: 1 and ending at residue 46 of SEQ ID NO: 1.

In some embodiments, the antisense polynucleotide includes 9 or fewer (e.g., 9, 8, 7 6, 5, 4, 3, 2, or 1) nucleotide mismatches relative to the region beginning at residue 17 of SEQ ID NO: 1 and ending at residue 46 of SEQ ID NO: 1. In some embodiments, the antisense polynucleotide includes 6 or fewer nucleotide mismatches relative to the region beginning at residue 17 of SEQ ID NO: 1 and ending at residue 46 of SEQ ID NO: 1. In some embodiments, the antisense polynucleotide includes 3 or fewer nucleotide mismatches relative to the region beginning at residue 17 of SEQ ID NO: 1 and ending at residue 46 of SEQ ID NO: 1.

In some embodiments, the antisense polynucleotide has a nucleic acid sequence that is at least 85% (e.g., 85%, 86%, 87%, 88%, 89%, 90%, 91%, 92%, 93%, 94%, 95%, 96%, 97%, 98%, 99%, or 99.9%) identical to the nucleic acid sequence of SEQ ID NO: 2. In some embodiments, the antisense polynucleotide has a nucleic acid sequence that is at least 90% identical to the nucleic acid sequence of SEQ ID NO: 2. In some embodiments, the antisense polynucleotide has a nucleic acid sequence that is at least 95% identical to the nucleic acid sequence of SEQ ID NO: 2. In some embodiments, the antisense polynucleotide has a nucleic acid sequence that is at least 96%, 97%, 98%, or 99% identical to the nucleic acid sequence of SEQ ID NO: 2. In some embodiments, the antisense polynucleotide has the nucleic acid sequence of SEQ ID NO: 2.

In some embodiments, the RNA molecule includes a U7 snRNA. In some embodiments, the U7 snRNA is located 3′ to the antisense polynucleotide.

In some embodiments, the U7 snRNA includes an optimized spliceosome-binding (Sm OPT) motif. In some embodiments, the Sm OPT motif has a nucleic acid sequence that is at least 85% (e.g., 85%, 86%, 87%, 88%, 89%, 90%, 91%, 92%, 93%, 94%, 95%, 96%, 97%, 98%, 99%, or 99.9%) identical to the nucleic acid sequence of SEQ ID NO: 5. In some embodiments, the Sm OPT motif has a nucleic acid sequence that is at least 90% identical to the nucleic acid sequence of SEQ ID NO: 5. In some embodiments, the Sm OPT motif has a nucleic acid sequence that is at least 95% identical to the nucleic acid sequence of SEQ ID NO: 5. In some embodiments, the Sm OPT motif has a nucleic acid sequence that is at least 96%, 97%, 98%, or 99% identical to the nucleic acid sequence of SEQ ID NO: 5. In some embodiments, the Sm OPT motif has the nucleic acid sequence of SEQ ID NO: 5.

In some embodiments, the U7 snRNA includes a U7 stem loop. In some embodiments, the U7 stem loop has a nucleic acid sequence that is at least 85% (e.g., 85%, 86%, 87%, 88%, 89%, 90%, 91%, 92%, 93%, 94%, 95%, 96%, 97%, 98%, 99%, or 99.9%) identical to the nucleic acid sequence of SEQ ID NO: 6. In some embodiments, the U7 stem loop has a nucleic acid sequence that is at least 90% identical to the nucleic acid sequence of SEQ ID NO: 6. In some embodiments, the U7 stem loop has a nucleic acid sequence that is at least 95% identical to the nucleic acid sequence of SEQ ID NO: 6. In some embodiments, the U7 stem loop has a nucleic acid sequence that is at least 96%, 97%, 98%, or 99% identical to the nucleic acid sequence of SEQ ID NO: 6.

In some embodiments, the U7 snRNA includes a U7 downstream region. In some embodiments, the U7 downstream region has a nucleic acid sequence that is at least 85% (e.g., 85%, 86%, 87%, 88%, 89%, 90%, 91%, 92%, 93%, 94%, 95%, 96%, 97%, 98%, 99%, or 99.9%) identical to the nucleic acid sequence of SEQ ID NO: 7. In some embodiments, the U7 downstream region has a nucleic acid sequence that is at least 90% identical to the nucleic acid sequence of SEQ ID NO: 7. In some embodiments, the U7 downstream region has a nucleic acid sequence that is at least 95% identical to the nucleic acid sequence of SEQ ID NO: 7. In some embodiments, the U7 downstream region has a nucleic acid sequence that is at least 96%, 97%, 98%, or 99% identical to the nucleic acid sequence of SEQ ID NO: 7.

In some embodiments, the U7 snRNA has a nucleic acid sequence that is at least 85% (e.g., 85%, 86%, 87%, 88%, 89%, 90%, 91%, 92%, 93%, 94%, 95%, 96%, 97%, 98%, 99%, or 99.9%) identical to the nucleic acid sequence of SEQ ID NO: 8. In some embodiments, the U7 snRNA has a nucleic acid sequence that is at least 90% identical to the nucleic acid sequence of SEQ ID NO: 8. In some embodiments, the U7 snRNA has a nucleic acid sequence that is at least 95% identical to the nucleic acid sequence of SEQ ID NO: 8. In some embodiments, the U7 snRNA has a nucleic acid sequence that is at least 96%, 97%, 98%, or 99% identical to the nucleic acid sequence of SEQ ID NO: 8. In some embodiments, the U7 snRNA has the nucleic acid sequence of SEQ ID NO: 8.

In some embodiments, the antisense polynucleotide is an antisense RNA (asRNA), a short interfering RNA (siRNA), a short hairpin RNA (shRNA), a micro RNA (miRNA), or an antisense oligonucleotide (ASO).

In another aspect, the disclosure provides a composition including the transgene or RNA molecule of any of the foregoing aspects, wherein the composition is a liposome, vesicle, synthetic vesicle, exosome, synthetic exosome, dendrimer, or nanoparticle.

In another aspect, the disclosure provides a plasmid including or encoding the transgene or RNA molecule of any of the foregoing aspects. In another aspect, the disclosure provides a non-viral vector including or encoding the transgene or RNA molecule of any of the foregoing aspects. In another aspect, the disclosure provides a viral vector including or encoding the transgene or RNA molecule of any of the foregoing aspects.

In some embodiments of the foregoing aspect, the plasmid, non-viral vector, or viral vector includes one or more of the RNA molecules. In some embodiments, the plasmid, non-viral vector, or viral vector includes two of the RNA molecules.

In some embodiments, the two RNA molecules are oriented bidirectionally.

In some embodiments, the viral vector is selected from the group consisting of adeno-associated virus (AAV), adenovirus, lentivirus, retrovirus, poxvirus, baculovirus, herpes simplex virus, vaccinia virus, and a synthetic virus.

In some embodiments, the viral vector is an AAV. In some embodiments, the AAV is a recombinant AAV (rAAV), a single-stranded rAAV, or a self-complementary recombinant AAV (scAAV). In some embodiments, the AAV is a single-stranded rAAV. In some embodiments, the AAV is a scAAV.

In some embodiments, the AAV includes capsid proteins from an AAV having a serotype selected from the group consisting of AAV1, AAV2, AAV3, AAV4, AAV5, AAV6, AAV7, AAV8, AAV9, AAVrh10, and AAVrh74.

In some embodiments, the viral vector is a pseudotyped AAV. In some embodiments, the pseudotyped AAV is AAV2/8 or AAV2/9. In some embodiments, the pseudotyped AAV is AAV2/8.

In some embodiments, expression of the transgene is regulated by an enhancer that promotes expression of the transgene in a muscle cell or neuron. In some embodiments, the enhancer is a muscle creatine kinase (MCK) enhancer, a desmin enhancer, a myosin light chain enhancer, a myosin heavy chain enhancer, a cardiac troponin C enhancer, a troponin I enhancer, a myoD gene family enhancer, an actin alpha enhancer, an actin beta enhancer, an actin gamma enhancer, or an enhancer within intron 1 of ocular paired like homeodomain 3. In some embodiments, the enhancer is a MCK enhancer.

In some embodiments, the MCK enhancer has a nucleic acid sequence that is at least 85% (e.g., 85%, 86%, 87%, 88%, 89%, 90%, 91%, 92%, 93%, 94%, 95%, 96%, 97%, 98%, 99%, or 99.9%) identical to the nucleic acid sequence of SEQ ID NO: 11. In some embodiments, the MCK enhancer has a nucleic acid sequence that is at least 90% identical to the nucleic acid sequence of SEQ ID NO: 11. In some embodiments, the MCK enhancer has a nucleic acid sequence that is at least 95% identical to the nucleic acid sequence of SEQ ID NO: 11. In some embodiments, the MCK enhancer has a nucleic acid sequence that is at least 96%, 97%, 98%, or 99% identical to the nucleic acid sequence of SEQ ID NO: 11. In some embodiments, the MCK enhancer has the nucleic acid sequence of SEQ ID NO: 11.

In some embodiments, the transgene is operably linked to a U7 promoter. In some embodiments, the 5′ end of the antisense polynucleotide is bound to the 3′ end of the U7 promoter.

In some embodiments, the U7 promoter has a nucleic acid sequence that is at least 85% (e.g., 85%, 86%, 87%, 88%, 89%, 90%, 91%, 92%, 93%, 94%, 95%, 96%, 97%, 98%, 99%, or 99.9%) identical to the nucleic acid sequence of SEQ ID NO: 4. In some embodiments, the U7 promoter has a nucleic acid sequence that is at least 90% identical to the nucleic acid sequence of SEQ ID NO: 4. In some embodiments, the U7 promoter has a nucleic acid sequence that is at least 95% identical to the nucleic acid sequence of SEQ ID NO: 4. In some embodiments, the U7 promoter has a nucleic acid sequence that is at least 96%, 97%, 98%, or 99% identical to the nucleic acid sequence of SEQ ID NO: 4. In some embodiments, the U7 promoter has the nucleic acid sequence of SEQ ID NO: 4.

In some embodiments, the transgene has a nucleic acid sequence that is at least 85% (e.g., 85%, 86%, 87%, 88%, 89%, 90%, 91%, 92%, 93%, 94%, 95%, 96%, 97%, 98%, 99%, or 99.9%) identical to the nucleic acid sequence of SEQ ID NO: 9. In some embodiments, the transgene has a nucleic acid sequence that is at least 90% identical to the nucleic acid sequence of SEQ ID NO: 9. In some embodiments, the transgene has a nucleic acid sequence that is at least 95% identical to the nucleic acid sequence of SEQ ID NO: 9. In some embodiments, the transgene has a nucleic acid sequence that is at least 96%, 97%, 98%, or 99% identical to the nucleic acid sequence of SEQ ID NO: 9. In some embodiments, the transgene has the nucleic acid sequence of SEQ ID NO: 9.

In some embodiments, the viral vector has a nucleic acid sequence that is at least 85% (e.g., 85%, 86%, 87%, 88%, 89%, 90%, 91%, 92%, 93%, 94%, 95%, 96%, 97%, 98%, 99%, or 99.9%) identical to the nucleic acid sequence of SEQ ID NO: 10. In some embodiments, the viral vector has a nucleic acid sequence that is at least 90% identical to the nucleic acid sequence of SEQ ID NO: 10. In some embodiments, the viral vector has a nucleic acid sequence that is at least 95% identical to the nucleic acid sequence of SEQ ID NO: 10. In some embodiments, the viral vector has a nucleic acid sequence that is at least 96%, 97%, 98%, or 99% identical to the nucleic acid sequence of SEQ ID NO: 10. In some embodiments, the viral vector has the nucleic acid sequence of SEQ ID NO: 10.

In another aspect, the disclosure provides a pharmaceutical composition including the composition or the viral vector of any of the foregoing aspects and a pharmaceutically acceptable carrier, diluent, or excipient.

In another aspect, the disclosure provides a method of treating a muscular dystrophy (e.g., DMD) in a human patient diagnosed as having a duplication in exon 2 of an endogenous DMD gene, the method including administering to the patient a therapeutically effective amount of the composition, viral vector, or pharmaceutical composition any of the foregoing aspects.

In another aspect, the disclosure provides a method of treating a disorder (e.g., DMD) in a human patient diagnosed as having a frameshift mutation, the method including administering to the patient a therapeutically effective amount of the composition, viral vector, or pharmaceutical composition any of the foregoing aspects.

In another aspect, the disclosure provides a method of treating a disorder (e.g., DMD) mediated by a frameshift mutation in a human patient, the method including administering to the patient a therapeutically effective amount of the composition, viral vector, or pharmaceutical composition any of the foregoing aspects.

In another aspect, the disclosure provides a method of treating a disorder (e.g., DMD) mediated by a frameshift mutation in a human patient, the method including administering to the patient a therapeutically effective amount of the composition, viral vector, or pharmaceutical composition any of the foregoing aspects.

In another aspect, the disclosure provides a method of treating a disorder (e.g., DMD) in a human patient diagnosed as overexpressing a protein of interest (e.g., dystrophin), the method including administering to the patient a therapeutically effective amount of the composition, viral vector, or pharmaceutical composition any of the foregoing aspects.

In another aspect, the disclosure provides a method of treating a disorder (e.g., DMD) mediated by overexpression of a protein of interest (e.g., dystrophin) in a human patient, the method including administering to the patient a therapeutically effective amount of the composition, viral vector, or pharmaceutical composition any of the foregoing aspects.

In another aspect, the disclosure provides a method of treating DMD in a human patient diagnosed as having a duplication in exon 2 of an endogenous DMD gene, the method including administering to the patient a therapeutically effective amount of the composition, viral vector, or pharmaceutical composition any of the foregoing aspects.

In another aspect, the disclosure provides a method of increasing expression of functional dystrophin protein in a human patient diagnosed as having a muscular dystrophy (e.g., DMD) and as having a duplication in exon 2 of an endogenous DMD gene, the method including administering to the patient a therapeutically effective amount of the composition, viral vector, or pharmaceutical composition any of the foregoing aspects.

In another aspect, the disclosure provides a method of inducing exon 2 skipping in a human patient diagnosed as having a muscular dystrophy (e.g., DMD) and as having a duplication in exon 2 of an endogenous DMD gene, the method including administering to the patient a therapeutically effective amount of the composition, viral vector, or pharmaceutical composition any of the foregoing aspects.

In another aspect, the disclosure provides a method of treating a muscular dystrophy (e.g., DMD) in a human patient diagnosed as having a frameshift mutation in any one of exons 1-4 of an endogenous DMD gene, the method including administering to the patient a therapeutically effective amount of the composition, viral vector, or pharmaceutical composition any of the foregoing aspects.

In another aspect, the disclosure provides a method of increasing expression of functional dystrophin protein in a human patient diagnosed as having a muscular dystrophy (e.g., DMD) and as having a frameshift mutation in any one of exons 1-4 of an endogenous DMD gene, the method including administering to the patient a therapeutically effective amount of the composition, viral vector, or pharmaceutical composition any of the foregoing aspects.

In another aspect, the disclosure provides a method of inducing activation of an internal ribosomal entry site within exon 5 of an endogenous DMD gene in a human patient, wherein the patient is diagnosed as having a muscular dystrophy (e.g., DMD) and as having a frameshift mutation in any one of exons 1-4 of the endogenous DMD gene, the method including administering to the patient a therapeutically effective amount of the composition, viral vector, or pharmaceutical composition any of the foregoing aspects.

In some embodiments of any of the foregoing aspects, the composition or viral vector is administered to the patient by way of intravenous, intrathecal, intracerebroventricular, intraparenchymal, intracisternal, intradermal, transdermal, parenteral, intramuscular, intranasal, subcutaneous, percutaneous, intratracheal, intraperitoneal, intraarterial, intravascular, inhalation, perfusion, lavage, or oral administration. In some embodiments, the composition or viral vector is administered to the patient intravenously.

In some embodiments of any of the foregoing aspects, the patient is a pediatric patient. In some embodiments, the patient is from about 6 months of age to about 14 years of age (e.g., about 6 months of age to about 13 years of age, about 6 months of age to about 12 years of age, about 6 months of age to about 11 years of age, about 6 months of age to about years of age, about 6 months of age to about 9 years of age, about 6 months of age to about 8 years of age, about 6 months of age to about 7 years of age, about 6 months of age to about 6 years of age, about 6 months of age to about 5 years of age, about 6 months of age to about 4 years of age, about 6 months of age to about 3 years of age, about 6 months of age to about 2 years of age, or about 6 months of age to about 1 year of age).

In some embodiments of any of the foregoing aspects, the patient is pre-ambulant. In some embodiments, the patient is ambulant.

In another aspect, the disclosure provides a kit including (i) the composition, viral vector, or pharmaceutical composition any of the foregoing aspects, and (ii) a package insert, wherein the package insert instructs a user of the kit to administer the composition, viral vector, or pharmaceutical composition to a human patient diagnosed as having a frameshift mutation.

In another aspect, the disclosure provides a kit including (i) the composition, viral vector, or pharmaceutical composition any of the foregoing aspects, and (ii) a package insert, wherein the package insert instructs a user of the kit to administer the composition, viral vector, or pharmaceutical composition to a human patient diagnosed as having overexpression of a protein of interest (e.g., dystrophin).

In another aspect, the disclosure provides a kit including (i) the composition, viral vector, or pharmaceutical composition any of the foregoing aspects, and (ii) a package insert, wherein the package insert instructs a user of the kit to administer the composition, viral vector, or pharmaceutical composition to a human patient diagnosed as having a muscular dystrophy (e.g., DMD).

In some embodiments of any of the foregoing aspects, the protein of interest is dystrophin.

In some embodiments of any of the foregoing aspects, the disorder is DMD or BMD. In some embodiments, the disorder is DMD. In some embodiments of any of the foregoing aspects, the muscular dystrophy is DMD or BMD. In some embodiments, the muscular dystrophy is DMD.

In some embodiments of any of the foregoing aspects, the patient is diagnosed as having a duplication in exon 2 of an endogenous DMD gene.

In some embodiments of any of the foregoing aspects, the patient is diagnosed as having a frameshift mutation in any one of exons 1-4 of an endogenous DMD gene. In some embodiments, the frameshift mutation is a frameshift mutation in exon 2 of an endogenous DMD gene.

### BRIEF DESCRIPTION OF THE DRAWINGS

FIG. 1 is a schematic drawing of an exemplary recombinant adeno-associated virus (rAAV) 8 (rAAV8) vector for the expression of a transgene including a BoxB RNA element and an antisense polynucleotide (e.g. an antisense ribonucleic acid (asRNA) complementary to a region between residues 17 and 46 of exon 2 of a human dystrophin (DMD) RNA transcript). From left to right, the shaded arrows and rectangles represent a rAAV8 encoding two bidrectionally oriented U7 small nuclear RNA expression casettes operatively linked to a muscle creatine kinase (MCK) enhancer; a stuffer (“synthetic 2”), and flanking rAAV2 inverted terminal repeat (ITR) sequences. Bidirectional arrows indicate the two U7 snRNA expression casettes, respectively, which include from 5′-to-3′ a BoxB RNA element, an antisense polynucleotide (e.g., a modified asRNA sequence targeting DMD exon 2), a U7 snRNA sequence, a U7 Sm binding protein (U7 Sm OPT) sequence, a U7 stem loop, and a U7 downstream element. Abbreviations: ESE, exonic splicing enhancer.

FIG. 2 is a schematic drawing of an exemplary U7 expression cassette containing an asRNA (“as”) complementary to a region between residues 17 and 46 of DMD exon 2, which includes a portion of exon 2 and the ESE region. From left to right, the shaded rectangles represent the nucleic acid sequence encoding a U7 promoter (U7-P) operatively linked to a U7 small nuclear RNA (snRNA) construct containing an asRNA targeting DMD exon 2, a U7 snRNA, a Sm binding protein sequence (Sm), and a U7 hairpin (hairpin; e.g., a U7 stem loop and a U7 downstream element).

FIG. 3 is a reverse transcription polymerase chain reaction (RT-PCR) illustrating the U7 snRNA vector approach to exon skipping in transduced Dup2 immortalized human fibromyoblasts using the rAAV8, as described in FIG. 1, with or without a BoxB RNA element, respectively, that encodes an exon 2-targeted U7 snRNA, as described in FIG. 2. A U7 snRNA expression cassette cloned into an AAV plasmid was used as a carrier to target the pre-messenger RNA of DMD exon 2. This U7 snRNA was composed of a U7 stem loop used for nucleocytoplasmic export, a U7 Sm OPT recognition sequence to bind Sm proteins for efficient assembly between the U7 snRNA and the target pre-mRNA, an asRNA to target a pre-mRNA of DMD exon 2, and a BoxB RNA element to enhance expression of the asRNA. The vector in lane 3 indicates a vector genome encoding a U7 snRNA with a BoxB RNA element. The vectors in lane “AAV-DMD-Ex2,” 1, 5, and 6 indicate vector genomes encoding a U7 snRNA without a BoxB RNA element, respectively.

FIG. 4 is a graph showing the relative amount of guide RNA (e.g., asRNA) expression level in host cells transduced with an rAAV8, as described in FIG. 1, with or without a BoxB RNA element, respectively, that encode an exon 2-targeted U7 snRNA, as described in FIG. 2.

### DEFINITIONS

As used herein, the term “about” refers to a value that is within 10% above or below the value being described.

As used herein, “activity” refers to form(s) of a nucleic acid or polypeptide which retains a biological activity of the native or naturally occurring nucleic acid or polypeptide, respectively, wherein “biological” activity refers to a biological function (e.g., splicing) caused by a native or naturally occurring nucleic acid or polypeptide, respectively.

As used herein, “administration” refers to providing or giving a subject a therapeutic agent (e.g., an inhibitory agent) by any effective route. Exemplary routes of administration are described herein and below (e.g., intracerebroventricular (ICV) injection, intrathecal (IT) injection, intraparenchymal (IP) injection, intravenous (IV) injection, and stereotactic injection). Administration may be systemic or local.

As used herein, the term “anneal” refers to the formation of a stable duplex of nucleic acids by way of hybridization mediated by inter-strand hydrogen bonding, for example, according to Watson-Crick base pairing. The nucleic acids of the duplex may be, for example, at least 50% complementary to one another (e.g., about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% complementary to one another. The “stable duplex” formed upon the annealing of one nucleic acid to another is a duplex structure that is not denatured by a stringent wash. Exemplary stringent wash conditions are known in the art and include temperatures of about 5° C. less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M, 0.1 M, 0.09 M, 0.08 M, 0.07 M, 0.06 M, 0.05 M, 0.04 M, 0.03 M, 0.02 M, 0.01 M, or less).

As used herein, the terms “antisense oligonucleotide” and “ASO” refer to a single-stranded oligonucleotide sequence that contains one or more modified nucleosides or nucleotides and is capable of (i) annealing to a target RNA transcript, thereby forming a nucleic acid duplex, in particular to a contiguous sequence on a target nucleic acid, and (ii) masking a splicing reaction by said annealing. In some embodiments, the ASO includes locked nucleic acid antisense oligonucleotides or alternative modifications.

As used herein, the terms “antisense RNA” and “asRNA” refer to a single-stranded cis-natural antisense transcript that has transcript complementary to other endogenous RNA transcripts and is capable of (i) annealing to a target RNA transcript, thereby forming a nucleic acid duplex, in particular to a contiguous sequence on a target nucleic acid, and (ii) masking a splicing reaction by said annealing.

As used herein, a “combination therapy” means that two (or more) different agents or treatments are administered to a subject as part of a defined treatment regimen for a particular disease or condition (e.g., DMD). The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be affected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the term “BoxB RNA element” refers to a bacteriophage λ (e.g., phage p22 and phi21) N-protein NutboxB (“BoxB”) ribonucleic acid (RNA) stem loop (e.g., an RNA hairpin). Such BoxB RNA elements have intramolecular base pairing, also known as a hairpin or hairpin loop, that occurs when two regions of the same RNA strand base-pair to form a double helix that ends in an unpaired loop. The resulting hairpin loop is a secondary structure of RNA, which may direct RNA folding, protect structural stability for messenger RNA (mRNA), provide recognition sites for RNA binding proteins, or serve as a substrate for enzymatic reactions, among other functions. As described herein a BoxB RNA element may refer to an RNA sequence whose nucleic acid sequence comprises or consists of a nucleic acid sequence of a naturally occurring wild-type bacteriophage λ N-protein NutboxB RNA stem loop as well as variants thereof. Bacteriophage λ BoxB has NCBI Macromolecular Structures Resource Group (MMBD) ID NO: 49478. An exemplary BoxB nucleic acid sequence is provided in SEQ ID NO: 3. As used herein, a BoxB RNA element may refer to an RNA sequence whose nucleic sequence is at least 75% (e.g., at least 76%, 77%, 78%, 79%, 80%, 81%, 82%, 83%, 84%, 85%, 86%, 87%, 88%, 89%0, 90%, 91%, 92%, 93%, 94%, 95%, 96%, 97%, 98%, 99%, or 99.9%) identical to the nucleic acid sequence of SEQ ID NO: 3.

As used herein, the term “DMD” refers to the gene for dystrophin. As used herein, the term DMD encompasses full-length, unprocessed DMD, as well as any form of DMD resulting from processing in the cell, as well as any naturally occurring variants of DMD (e.g., splice variants or allelic variants). Human DMD has NCBI Gene ID: 1756. An exemplary wild-type human DMD isoform DP427p1 nucleic acid sequence is provided in NCBI RefSeq Acc. No. NM_004009.3, and an exemplary wild-type human DMD isoform DP427p1 amino acid sequence is provided in NCBI RefSeq Acc. No. NP_004000.1. An exemplary wild-type human DMD isoform Dp427p2 nucleic acid sequence is provided in NCBI RefSeq Acc. No NM_004010.3, and an exemplary wild-type human DMD isoform Dp427p2 amino acid sequence is provided in NCBI RefSeq Acc. NP_004001.1. An exemplary wild-type human DMD isoform Dp71 nucleic acid sequence is provided in NCBI RefSeq Acc. No. NM_004015.3, and an exemplary wild-type human DMD isoform Dp71 amino acid sequence is provided in NCBI RefSeq Acc. NP_004006.1. An exemplary wild-type human DMD isoform Dp71b nucleic acid sequence is provided in NCBI RefSeq Acc. No. NM_004016.3, and an exemplary wild-type human DMD isoform Dp71b amino acid sequence is provided in NCBI RefSeq Acc. NP_004007.1. An exemplary wild-type human DMD isoform Dp71a nucleic acid sequence is provided in NCBI RefSeq Acc. No. NM_004017.3, and an exemplary wild-type human DMD isoform Dp71a amino acid sequence is provided in NCBI RefSeq Acc. NP_004008.1. An exemplary wild-type human DMD isoform Dp71ab nucleic acid sequence is provided in NCBI RefSeq Acc. No. NM_004018.3, and an exemplary wild-type human DMD isoform Dp71ab amino acid sequence is provided in NCBI RefSeq Acc. NP_004009.1. An exemplary wild-type human DMD isoform Dp40 nucleic acid sequence is provided in NCBI RefSeq Acc. No. NM_004019.3, and an exemplary wild-type human DMD isoform Dp40 amino acid sequence is provided in NCBI RefSeq Acc. NP_004010.1.

In the practice of the methods of the presently disclosed subject matter, an “effective amount” of any one of the compounds or a combination of any of the compounds or a pharmaceutically acceptable salt thereof, is administered via any of the usual and acceptable methods known in the art, either singly or in combination.

As used herein, the term “endogenous” describes a molecule (e.g., a metabolite, polypeptide, nucleic acid, or cofactor) that is found naturally in a particular organism (e.g., a human) or in a particular location within an organism (e.g., an organ, a tissue, or a cell, such as a human cell).

As used herein, the term “exon” refers to a region within the coding region of a gene, the nucleotide sequence of which determines the amino acid sequence of the corresponding protein. The term exon also refers to the corresponding region of the RNA transcribed from a gene. A gene, as defined above, may contain, for instance, a minimum of three exons separated by intervening introns. Exons are transcribed into pre-mRNA and may be included in the mature mRNA depending on the alternative splicing of the gene. Exons that are included in the mature mRNA following processing are translated into protein, wherein the sequence of the exon determines the amino acid composition of the protein.

As used herein, the term “exon skipping” refers to a form of alternative splicing wherein one or more exons are excluded from the resulting mature mRNA. Exon skipping may occur as a result of endogenous regulation or may be induced in a pre-determined manner by the presence of a polynucleotide (e.g., an antisense polynucleotide).

As used herein, the term “frameshift” refers to a change in how a ribosome defines a codon within a gene, and therefore defined the reading frame of translation. The identity of each amino acid in a protein is determined by a three-nucleotide codon, which is defined by the ribosome through binding of the anticodon of a tRNA to its complementary sequence on an mRNA. Therefore, any occurrence of a frameshift may alter the amino acid sequence of the translated protein both at and downstream of the location of the frameshift. As used herein, the term “genetic frameshift” refers to any mutation that results in a frameshift. A deletion or insertion within an exon, wherein the deletion or insertion is not a multiple of three nucleotides, may result in a frameshift. Additionally, a duplication or deletion of an entire exon, wherein the number of nucleotides in the exon is not a multiple of three, may result in a frameshift. In some embodiments, a frameshift mutation refers to a frameshift mutation in any one of exons 1-4 of the endogenous DMD gene

As used herein, the term “gene” refers to a region of DNA that encodes a protein. A gene may include regulatory regions and a protein-coding region. In some embodiments, a gene includes two or more introns and three or more exons, wherein each intron forms an intervening sequence between two exons.

As used herein, the term “antisense polynucleotide” refers to a RNA, such as an antisense RNA (asRNA), small interfering RNA (siRNA), micro RNA (miRNA), short hairpin RNA (shRNA), or an antisense oligonucleotide (ASO) that suppresses the endogenous function of a target RNA transcript, for example, by way of (i) annealing to the target RNA transcript, thereby forming a nucleic acid duplex; and (ii) masking the binding of a specific splice-related region (e.g., an exonic splicing enhancer, splice acceptor, or splice donor) during the splicing reaction. Antisense polynucleotides as described herein may be provided to a patient, such as a human patient having a muscular dystrophy (e.g., DMD) described herein, in the form of, for example, a single- or double-stranded oligonucleotide, or in the form of a vector (e.g., a viral vector) containing a transgene encoding the antisense polynucleotide (e.g., asRNA). Exemplary antisense polynucleotide platforms are described, for example, in Lam et al., Mol. Ther. Nucleic Acids 4:e252 (2015); Rao et al., Adv. Drug Deliv. Rev. 61:746-769 (2009); and Borel et al., Mol. Ther. 22:692-701 (2014), the disclosures of each of which are incorporated herein by reference in their entirety. In some embodiments, the antisense polynucleotide is an asRNA.

As used herein, the term “IRES” refers to an internal ribosome entry site. In general, an IRES sequence is a feature that allows eukaryotic ribosomes to bind an mRNA transcript and begin translation without binding to a 5′ capped end. An mRNA containing an IRES sequence produces two translation products, one initiating form the 5′ end of the mRNA and the other from an internal translation mechanism mediated by the IRES.

As used herein, the “length” of a nucleic acid refers to the linear size of the nucleic acid as assessed by measuring the quantity of nucleotides from the 5′ to the 3′ end of the nucleic acid. Exemplary molecular biology techniques that may be used to determine the length of a nucleic acid of interest are known in the art.

By “muscular dystrophy” is meant the group of muscle diseases that weaken the musculoskeletal system and hamper locomotion. Muscular dystrophies are characterized by progressive deterioration of muscle function (e.g., weakness), defects in muscle proteins, and death of muscle cells and tissue.

Some types of muscular dystrophy are characterized as dystrophinopathy, which includes a spectrum of muscle diseases in which there is insufficient dystrophin protein produced in the muscle cells, resulting in instability in the structure of the muscle cell membrane. Non-limiting examples of dystrophinopathies include Duchenne muscular dystrophy (DMD) and Becker muscular dystrophy (BMD, also known as Benign pseudohypertrophic muscular dystrophy). DMD and BMD are X-linked recessive diseases caused by mutations in the dystrophin gene (DMD), which encodes the protein dystrophin. DMD is more severe than BMD because typically no dystrophin protein is produced in the affected muscle cells in DMD, whereas in BMD, defective dystrophin is produced. Other examples of muscular dystrophies include but are not limited to congenital muscular dystrophy, facioscapulohumeral muscular dystrophy, limb-girdle muscular dystrophy, myotonic muscular dystrophy, and oculopharyngeal muscular dystrophy.

As used herein, the term “mutation” refers to any change in the sequence of a gene, such that the sequence is not identical to that of the wild type gene. A mutation may be selected from the group including a single nucleotide point mutation that results in a premature termination codon, a single nucleotide insertion, a single nucleotide deletion, the insertion of two or more contiguous nucleotides, the deletion of two or more contiguous nucleotides, the duplication of a contiguous region within a gene (e.g., an exon e.g., DMD exon 2), or the deletion of a contiguous region within a gene. A mutated gene may include a single mutation, or multiple mutations. A mutation may occur in any region of the gene.

As used herein, the terms “nucleic acid molecule,” “nucleic acid,” and “polynucleotide” are used interchangeably and refer to polymers of nucleotides of any length. Examples of polynucleotides are DNA polynucleotides and RNA polynucleotides. All nucleic acid sequences herein are written in the 5′-to-3′ direction and are to be construed accordingly.

As used herein, the term “operably linked” in the context of a nucleic acid refers to a nucleic acid that is placed into a structural or functional relationship with another nucleic acid. For example, one segment of DNA may be operably linked to another segment of DNA if they are positioned relative to one another on the same contiguous DNA molecule and have a structural or functional relationship, such as a promoter or enhancer that is positioned relative to a coding region so as to facilitate transcription of the coding region. In other examples, the operably linked nucleic acids are not contiguous, but are positioned in such a way that they have a functional relationship with each other as nucleic acids or as proteins that are expressed by them. Enhancers, for example, do not have to be contiguous. Linking may be accomplished by ligation at convenient restriction sites or by using synthetic oligonucleotide adaptors or linkers. In some embodiments, a transgene described herein is operably linked to a muscle creatine kinase (MCK) enhancer. In some embodiments, a transgene described herein is operably linked to a U7 promoter.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

100 multiplied by (the fraction X/Y)
where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

The term “complementarity sufficient to hybridize,” as used herein, refers to a nucleic acid sequence or a portion thereof that need not be fully complementary (e.g., 100% complementary) to a target region or a nucleic acid sequence or a portion thereof that has one or more nucleotide mismatches relative to the target region but that is still capable of hybridizing to the target region under specified conditions. For example, the nucleic acid may be, e.g., 95% complementary, 90%, complementary, 85% complementary, 80% complementary, 75% complementary, 70% complementary, 65% complementary, 60% complementary, 55% complementary, 50% complementary, or less, but still form sufficient base pairs with the target so as to hybridize across its length.

As used herein, the term “hybridize” refers to the formation of a stable duplex of nucleic acids by way of annealing mediated by inter-strand hydrogen bonding, for example, according to Watson-Crick base pairing. The nucleic acids of the duplex may be, for example, at least 50% complementary to one another (e.g., about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100%) complementary to one another. The “stable duplex” formed upon the hybridization of one nucleic acid to another is a duplex structure that is not denatured by a stringent wash. Exemplary stringent wash conditions are known in the art and include temperatures of about 5° C. less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M, 0.1 M, 0.09M, 0.08M, 0.07M, 0.06M, 0.05 M, 0.04M, 0.03 M, 0.02M, 0.01 M, or less). The complementarity of the nucleic acids of the duplex may be low overall (e.g., less than 95%, less than 90%, less than 85%, less than 80%, less than 70%, less than 60%, less than 50%) but there may be segments of the nucleic acid that are contiguous and fully complementary to an equal-length segment of the target that, in the duplex form, allow for hybridizing across the target's length (e.g., the overall complementarity may be low, but there may be segments of at least 10 contiguous nucleotides, at least 11 contiguous nucleotides, at least 12 contiguous nucleotides, at least 13 contiguous nucleotides, at least 14 contiguous nucleotides, at least 15 contiguous nucleotides, at least 20 contiguous nucleotides, at least 25 contiguous nucleotides, at least 30 contiguous nucleotides) that are fully complementarity to an equal-length segment of the target, thus facilitating hybridization across the target's length.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “pharmaceutical composition,” as used herein, represents a composition containing a nucleic acid described herein, formulated with a pharmaceutically acceptable excipient, and manufactured or sold with the approval of a governmental regulatory agency as part of a therapeutic regimen for the treatment of disease in a subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “plasmid” refers to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

The term “promoter,” as used herein, refers to a region within the regulatory region of a gene that enables initiation of the transcription of a gene into a messenger RNA, wherein transcription is initiated with the binding of a RNA polymerase on or nearby the promoter. In some embodiments, the promoter is a U7 promoter.

The term “reading frame” refers to how a ribosome defines a codon within a gene, as determined by binding of a tRNA to a three-nucleotide codon during translation of an mRNA. When a reading frame is “restored,” this indicates that the reading frame had first been first altered by a frameshift or nonsense mutation, and subsequently returned to the original reading frame by some means.

By a “reference” is meant any useful reference used to compare protein or nucleic acid (e.g., mRNA) levels related to muscular dystrophies (e.g., DIMD). The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a muscular dystrophy (e.g., DMD); a sample from a subject that is diagnosed with a muscular dystrophy (e.g., DMD); a sample from a subject that has been treated for a muscular dystrophy (e.g., DMD); or a sample of a purified protein (e.g., dystrophin) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a muscular dystrophy (e.g., DMD). In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

As used herein, the terms “subject” and “patient” are interchangeable and refer to an organism that receives treatment for a particular disease or condition as described herein. In preferred embodiments, the subject is a human.

As used herein, the terms “transduction” and “transduce” refer to a method of introducing a viral vector construct or a part thereof into a cell and subsequent expression of a transgene or RNA molecule encoded by the vector construct or part thereof in the cell.

As used herein, the term “transcription regulatory element” refers to a nucleic acid that controls, at least in part, the transcription of a gene of interest. Transcription regulatory elements may include promoters, enhancers, and other nucleic acids (e.g., polyadenylation signals) that control or help to control gene transcription. Examples of transcription regulatory elements are described, for example, in Goeddel, Gene Expression Technology: Methods in Enzymology 185 (Academic Press, San Diego, CA, 1990). In some embodiments, a transcription regulatory element of a composition herein is a MCK enhancer. In some embodiments, a transcription regulatory element of a composition herein is U7 promoter.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, diethylaminoethyl (DEAE)-dextran transfection, NUCLEOFECTION™, squeeze-poration, sonoporation, optical transfection, MAGNETOFECTION™, impalefection, and the like.

As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the term “U7 snRNA” refers to a polymerase II transcript involved in the 3′-end processing of nonpolyadenylated histone mRNAs, which is required for S phase-specific gene expression. As used herein, U7 snRNA may be used as a carrier to target pre-messenger RNA of a target gene (e.g., DMD) or a portion thereof (e.g., an ESE, a splice acceptor (SA), a splice donor (SD), an exon (e.g., exon 2), or a portion thereof) and may be composed of: a BoxB RNA element or a variant thereof (e.g., used to enhance expression of an antisense polynucleotide), an antisense polynucleotide or a modified variant thereof (e.g., used to interfere with the function or expression of a target sequence), a U7 stem loop (e.g., used for nucleocytoplasmic export), and/or a Sm binding protein sequence recognition sequence (e.g., used to bind Sm proteins for efficient assembly between the U7 snRNA and the target pre-mRNA).

As used herein, the term “vector” includes a nucleic acid vector, e.g., a DNA vector, such as a plasmid, a RNA vector, virus, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/011026; incorporated herein by reference as it pertains to vectors suitable for the expression of a gene of interest. Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of transgenes as described herein include plasmids that contain regulatory sequences, such as promoter and enhancer regions, which direct gene transcription. Other useful vectors for expression of transgenes contain polynucleotide sequences that enhance the rate of translation of these genes or improve the stability or nuclear export of the mRNA that results from gene transcription. These sequence elements include, e.g., 5′ and 3′ untranslated regions, an IRES, and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, or zeocin.

As used herein, the terms “5′ mutation of the DMD gene” and “Δ5′” refer to a mutation within or affecting exon 1, 2, 3, or 4 of the DMD gene. In some embodiments, the “5′ mutation of the DMD gene” is a duplication in exon 2 of an endogenous DMD gene. In some embodiments, the “5′ mutation of the DMD gene” is a frameshift mutation in any one of exons 1-4 of an endogenous DMD gene.

### DETAILED DESCRIPTION

The compositions and methods described herein are useful for mediating targeted exon skipping and for treating disorders associated with mutations in the dystrophin gene (DMD), such as muscular dystrophies (e.g., Duchenne muscular dystrophy (DMD)). The compositions described herein include transgenes that include a BoxB RNA element and an antisense polynucleotide (e.g., a small antisense RNA) that masks the binding of specific exonic splicing enhancer (ESE) regions and/or splice sites during the splicing reaction. This masking provides an important physiological benefit, including disrupting the splicing and translation of operably linked, mutated transcripts, mutated upstream transcripts, or mutated downstream transcripts. Without being limited by mechanism, the compositions described herein may ameliorate this pathology by diminishing the expression of RNA transcripts harboring mutations (e.g., a 5′ mutation of the DMD gene). For example, the compositions and methods described herein may be used to treat disorders, such as DMD, associated with a frameshift mutation or an exon duplication in the endogenous DMD gene. Moreover, the targeted exon skipping described herein may be enhanced by the inclusion of a BoxB RNA element, due to its ability to enhance the expression of the antisense polynucleotide.

The antisense polynucleotide described herein may be in any of a variety of forms, such as antisense RNA (asRNA), short interfering RNA (siRNA), short hairpin RNA (shRNA), micro RNA (miRNA), or antisense oligonucleotide (ASO). The antisense polynucleotides described herein may additionally be incorporated into a U7 small nuclear ribonucleic acid (snRNA) cassette and/or encoded by a vector, such as a viral vector. For example, described herein are adeno-associated viral (AAV) vectors, such as pseudotyped AAV vectors (e.g., an AAV2/8 vector) containing transgenes encoding antisense polynucleotide constructs that mask the binding of an ESE or one or more splice sites (e.g., SA, SD, or a combination thereof) during the splicing reaction.

The use of U7 snRNA for the delivery of DMD exon 2-targeted antisense polynucleotides, including interfering RNAs designed to anneal to splicing motifs at the ESE and/or intron:exon boundaries (e.g., splice acceptor (SA) or splice donor (SD) sites) for altering the expression of DMD exon 2 has been tested. Previous attempts to induce skipping of DMD exon 2 by targeting the center of the exon with asRNA have had limited success, if any, and the presently disclosed subject matter is based, at least in part, on the discovery that the combination of a BoxB RNA element together with an antisense polynucleotide (e.g., a small antisense RNA that elicits RNA interference through targeting a region that is between +17 and +46 of DMD exon 2, which includes the ESE), leads to a surprisingly superior ability to induce exon 2 skipping.

The compositions and methods described herein provide, among other benefits, the advantageous feature of being able to mediate efficacious skipping of DMD exon 2. This property is particularly beneficial in view of the prevalence of 5′ mutations of the DMD gene in mammalian genomes, such as in the genomes of human patients with DMD. Using the compositions and methods described herein, the expression of RNA transcripts that contain mutations can be diminished, while preserving the expression of important healthy RNA transcripts and their encoded protein products.

The sections that follow provide a description of exemplary BoxB RNA elements, antisense polynucleotides (e.g., interfering RNA), and U7 snRNA constructs that may be used in conjunction with the compositions and methods described herein, as well as a description of vectors encoding such constructs and methods that may be used to treat disorders associated with muscular dystrophies, such as DMD.

### Methods of Treating Muscular Dystrophies

Muscular dystrophies are a group of inherited disorders characterized by progressive muscle weakness. Deletions and point mutations in the DMD gene cause either the severe progressive myopathy DMD or the milder Becker muscular dystrophy (BMD), depending on whether the translational reading frame is lost or maintained. Specifically, the phenotype generally depends upon whether the mutation results in the complete absence of the protein product dystrophin (e.g., in DMD) or preserves a reading frame that allows translation of a partially functional dystrophin protein (e.g., in BMD) (e.g., see Monaco et al. Trends Biochem Sci, 14: 412-415 (1989)).

Many clinical cases of DMD are linked to deletion mutations in the DMD gene. For example, DMD patients may have a mutation within or affecting exon 1, 2, 3, or 4 of the DMD gene (e.g., a 5′ mutation of the DMD gene). In this clinical population, the targeted skipping of exon 2 and putative activation of an inducible IRES in exon 5 of the DMD gene may provide useful, as such activation generates a functional N-terminally truncated dystrophin isoform. In some embodiments, the present disclosure provides compositions and methods for treating DMD patients having one or more 5′ mutations of the DMD gene (e.g., a frameshift mutation in any one of exons 1-4 of an endogenous DMD gene).

In contrast to deletion mutations, DMD exon duplications account for around 5% of disease-causing mutations, as evaluated in unbiased samples of dystrophinopathy patients (e.g., see Dent et al., Am. J. Med. Genet, 734(3): 295-298 (2005)). In this clinical population, the targeted skipping of exon 2 provides therapeutic value, as one or both copies may be skipped. In some embodiments, the present disclosure provides compositions and methods for treating DMD patients having a duplication of exon 2 of the DMD gene.

As described herein, DMD exon 2 refers to a nucleic acid sequence having the nucleic acid sequence of SEQ ID NO: 1, shown below:

ATGAAAGAGAAGATGTTCAAAAGAAAACATT
CACAAAATGGGTAAATGCACAATTTTCTAAG.

Using the compositions and methods described herein, a patient experiencing a muscular dystrophy, such as DMD, can be administered a transgene that includes a BoxB RNA element and an antisense polynucleotide, or a vector encoding the same, so as to mask the binding of an ESE or splice site (e.g., SA, SD, or a combination thereof) during the splicing reaction. Without being limited by mechanism, this masking provides the beneficial effect of mediating altered splicing of DMD mRNA transcripts, thereby skipping exons that may, themselves, harbor mutations (e.g., exon duplications) or whose skipping may restore the translational reading frame such that a partially functional protein is generated. In some embodiments, skipping exons may result in the activation of the inducible IRES in exon 5 of the DMD gene, thereby generating a functional N-terminally truncated dystrophin isoform.

In some embodiments, the disclosure provides a method of treating a disorder (e.g., DMD) in a human patient diagnosed as having a frameshift mutation (e.g., a frameshift mutation in any one of exons 1-4 of an endogenous DMD gene e.g., a frameshift mutation in exon 2 of an endogenous DMD gene).

In some embodiments, the disclosure provides a method of treating a disorder (e.g., DMD) mediated by a frameshift mutation (e.g., a frameshift mutation in any one of exons 1-4 of an endogenous DMD gene e.g., a frameshift mutation in exon 2 of an endogenous DMD gene) in a human patient.

In some embodiments, the disclosure provides a method of treating a disorder in a human patient diagnosed as overexpressing a protein of interest (e.g., dystrophin).

In some embodiments, the disclosure provides a method of treating a disorder mediated by overexpression of a protein of interest (e.g., dystrophin) in a human patient.

In some embodiments, administration of a composition described herein provides a method of treating DMD in a human patient diagnosed as having a duplication in exon 2 of an endogenous DMD gene.

In some embodiments, administration of a composition described herein provides a method of increasing expression of functional dystrophin protein in a human patient diagnosed as having DMD and as having a duplication in exon 2 of an endogenous DMD gene.

In some embodiments, administration of a composition described herein provides a method of inducing exon 2 skipping in a human patient diagnosed as having DMD and as having a duplication in exon 2 of an endogenous DMD gene.

In some embodiments, administration of a composition described herein provides a method of treating DMD in a human patient diagnosed as having a frameshift mutation in any one of exons 1-4 of an endogenous DMD gene.

In some embodiments, the patient is a pediatric patient.

In some embodiments, the patient is from about 6 months of age to about 14 years of age (e.g., about 6 months of age to about 13 years of age, about 6 months of age to about 12 years of age, about 6 months of age to about 11 years of age, about 6 months of age to about years of age, about 6 months of age to about 9 years of age, about 6 months of age to about 8 years of age, about 6 months of age to about 7 years of age, about 6 months of age to about 6 years of age, about 6 months of age to about 5 years of age, about 6 months of age to about 4 years of age, about 6 months of age to about 3 years of age, about 6 months of age to about 2 years of age, or about 6 months of age to about 1 year of age).

In some embodiments, the patient is pre-ambulant or ambulant.

### BoxB RNA Element

The transgenes described herein may include a BoxB RNA element or a variant thereof. A wild-type BoxB RNA element consists of 15 nucleotides, and folds into a hairpin structure that contains a pentaloop. As described herein, any suitable stem loop and/or pentaloop may be used. Without being bound by mechanism, the advantage of including a BoxB RNA element in a transgene or RNA molecule described herein is that the BoxB RNA element may increase the expression of an antisense polynucleotide that is also encoded by the transgene or RNA molecule.

In some embodiments, the BoxB RNA element has a nucleic acid sequence that is at least 85% (e.g., 86%, 87%, 88%, 89%, 90%, 91%, 92%, 93%, 94%, 95%, 96%, 97%, 98%, 99%, or 99.9%) identical to the nucleic acid sequence of SEQ ID NO: 3. In some embodiments, the BoxB RNA element has a nucleic acid sequence that is 86% identical to the nucleic acid sequence of SEQ ID NO: 3. In some embodiments, the BoxB RNA element has a nucleic acid sequence that is 87% identical to the nucleic acid sequence of SEQ ID NO: 3. In some embodiments, the BoxB RNA element has a nucleic acid sequence that is 88% identical to the nucleic acid sequence of SEQ ID NO: 3. In some embodiments, the BoxB RNA element has a nucleic acid sequence that is 89% identical to the nucleic acid sequence of SEQ ID NO: 3. In some embodiments, the BoxB RNA element has a nucleic acid sequence that is 90% identical to the nucleic acid sequence of SEQ ID NO: 3. In some embodiments, the BoxB RNA element has a nucleic acid sequence that is 91% identical to the nucleic acid sequence of SEQ ID NO: 3. In some embodiments, the BoxB RNA element has a nucleic acid sequence that is 92% identical to the nucleic acid sequence of SEQ ID NO: 3. In some embodiments, the BoxB RNA element has a nucleic acid sequence that is 93% identical to the nucleic acid sequence of SEQ ID NO: 3. In some embodiments, the BoxB RNA element has a nucleic acid sequence that is 94% identical to the nucleic acid sequence of SEQ ID NO: 3. In some embodiments, the BoxB RNA element has a nucleic acid sequence that is 95% identical to the nucleic acid sequence of SEQ ID NO: 3. In some embodiments, the BoxB RNA element has a nucleic acid sequence that is 96% identical to the nucleic acid sequence of SEQ ID NO: 3. In some embodiments, the BoxB RNA element has a nucleic acid sequence that is 97% identical to the nucleic acid sequence of SEQ ID NO: 3. In some embodiments, the BoxB RNA element has a nucleic acid sequence that is 98% identical to the nucleic acid sequence of SEQ ID NO: 3. In some embodiments, the BoxB RNA element has a nucleic acid sequence that is 99% identical to the nucleic acid sequence of SEQ ID NO: 3. In some embodiments, the BoxB RNA element has a nucleic acid sequence that is identical to the nucleic acid sequence of SEQ ID NO: 3.

In some embodiments, the BoxB RNA element is positioned 5′ to the antisense polynucleotide or the BoxB RNA element is positioned 3′ to the antisense polynucleotide. For example, in some embodiments, the BoxB RNA element is positioned 5′ to the antisense polynucleotide. In some embodiments, the BoxB RNA element is positioned 3′ to the antisense polynucleotide.

### Inducing Alternative RNA Splicing

The presently disclosed subject matter provides compositions and methods that induce alternative splicing, for instance, by way of exon skipping, in a gene by using interfering RNA (e.g., an asRNA) to mask the binding of an ESE region and/or masking the utilization of splice sites (e.g., a SA or a SD site) during the splicing reaction.

The presently disclosed subject matter uses targeted alternative splicing (for example, by way of exon skipping) during the splicing of a pre-mRNA to form an mRNA. If the desired splice pattern omits one or more exons endogenous to a given host gene, when the host gene mRNA is translated by the ribosome, the resulting protein may be truncated, such that the truncated protein lacks one or more exteins of the wild-type form of the protein (e.g., a functional N-terminally truncated dystrophin isoform).

In some embodiments, the presently disclosed subject matter provides a composition for inducing alternative splicing of an mRNA encoding a protein by providing a cell with an antisense polynucleotide (e.g., an asRNA) that is complementary to a portion of the endogenous RNA transcript including residues +17 to +46 of the human dystrophin exon 2, which includes the ESE region, relative to the nucleic acid sequence of SEQ ID NO: 1.

### Therapeutic Applications of Alternative Splicing

### (i) Alternative Splicing for Reading Frame Restoration

The ability to induce targeted alternative splicing resulting in the production of a selectively truncated protein has well documented therapeutic value. In some embodiments, alternative splicing is induced in the form of exon skipping. In some embodiments, alternative splicing (e.g., exon skipping) may be used to restore complete or partial function to a protein, wherein the gene encoding the protein contains a mutation relative to the wild-type form of the gene (e.g., a 5′ mutation of the DMD gene).

In some embodiments, a host may harbor a deleterious genetic mutation in an exon (e.g., exon 1, 2, 3, or 4, of the DMD gene), wherein targeted skipping of an exon (e.g., DMD exon 2) may allow for complete or partial restoration of the function of the protein. In some embodiments, the mutation is a point mutation, insertion, or deletion that results in either a premature termination codon or a frameshift. In these embodiments, induced alternative splicing results in the exclusion of DMD exon 2 from the mature RNA causing a frameshift in the DMD gene reading frame and inducing utilization of the IRES in exon 5 for translational initiation, thereby generating a functional N-terminally truncated dystrophin isoform.

In another example, a host may harbor a deleterious genetic mutation in which a region including one or more exons, or a portion of one or more exons, has been duplicated (e.g., DMD exon 2 duplication) resulting in a frameshift. Targeted alternative splicing of one or more additional exons, such that the removal of the duplicated exon (e.g., DMD exon 2) from the mRNA restores the downstream reading frame, may allow for complete or partial restoration of the function of the protein. In some embodiments, the mutation is a duplication of a region of a gene including an entire exon (e.g., DMD exon 2), wherein in the mutation results in a frameshift. In other related embodiments, the mutation is a duplication of a region of a gene including a portion of an exon, wherein in the mutation results in a frameshift. In these embodiments, induced alternative splicing of one or more exons, such that alternative splicing results in restoration of the downstream reading frame, may restore partial or complete function of the protein.

In some embodiments, alternative splicing (e.g., by way of exon skipping) may be used in the treatment of DMD. As discussed previously, DMD arises from frameshifting or nonsense mutations in the DMD gene that codes for the dystrophin protein. Alternative splicing induced by the compositions and methods described herein may restore the downstream reading frame in the mutated DMD gene, thus restoring partial function of the protein.

### (ii) Alternative Splicing for Reading Frame Disruption

In some embodiments, it may be therapeutically useful to produce a C-terminally truncated protein; for example, if a protein is overexpressed, if a protein contains a dominant negative mutation, or if a chromosomal aberration results in a fusion protein associated with disease. In such cases, alternative splicing may be used to disrupt the reading frame in order to include a premature termination codon in the mRNA transcript and produce an N-terminally truncated protein.

In some embodiments, alternative splicing may be used to disrupt the downstream reading frame and result in a premature stop codon in order to activate a downstream IRES. Alternative splicing to disrupt the reading frame would require skipping of one or more exons, wherein the total number of nucleotides in the one or more exons being skipped does not add up to a multiple of 3. Moreover, design of the alternative splicing construct should consider the downstream sequence such that the alternative splicing results in a premature termination codon at the desired location.

In some embodiments, alternative splicing (e.g., by way of exon skipping to disrupt the reading frame in order to include a premature termination codon) may be used in the treatment of DMD. As discussed previously, DMD arises from frameshifting or nonsense mutations in the DMD gene that codes for the dystrophin protein. Alternative splicing induced by the compositions and methods described herein may disrupt the downstream reading frame in the mutated DMD gene, thus activate a downstream IRES and restoring partial function of the protein.

### Interfering RNA

The RNA molecule described herein may be any small antisense RNA molecule (e.g., an interfering RNA molecule, e.g., that acts to mask the binding or the utilization of an ESE and/or one or more splice sites (e.g., SA, SD, or a combination thereof) or a combination thereof during the splicing reaction). For example, an interfering RNA molecule includes an asRNA, siRNA, shRNA, miRNA, or an ASO that targets DMD exon 2 ESE. An asRNA is a single-stranded cis-natural antisense transcript that has transcript complementary to other endogenous RNA transcripts and is capable of annealing to a target RNA transcript (e.g., thereby forming a nucleic acid duplex, in particular to a contiguous sequence on a target nucleic acid and masking a splicing reaction by said annealing).

An siRNA is a double-stranded RNA molecule that typically has a length of about 19-base pairs. An shRNA is a RNA molecule containing a hairpin turn that decreases function and/or expression of target genes via RNA interference (RNAi). shRNAs can be delivered to cells in the form of plasmids (e.g., viral or bacterial vectors), by transfection, electroporation, or transduction. A microRNA is a non-coding RNA molecule that typically has a length of about 22 nucleotides. miRNAs bind to target sites on mRNA molecules and silence the mRNA, e.g., by causing cleavage of the mRNA, destabilization of the mRNA, or inhibition of translation of the mRNA. An ASO is a single-stranded oligonucleotide sequence that contains one or more modified nucleosides or nucleotides and is capable of annealing to a target RNA transcript (e.g., thereby forming a nucleic acid duplex, in particular to a contiguous sequence on a target nucleic acid and masking a splicing reaction by said annealing).

In some embodiments, the antisense polynucleotide molecule decreases the function and/or activity of the DMD exon 2 ESE. In some embodiments, the antisense polynucleotide molecule decreases the function and/or activity of the DMD exon 2 SA. In some embodiments, the antisense polynucleotide molecule decreases the function and/or activity of the DMD exon 2 SD.

An antisense polynucleotide (e.g., interfering RNA molecule e.g., an siRNA, a shRNA, a miRNA, or an ASO) can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, or 2′-deoxyuridine. Without being bound by theory, it is believed that certain modifications can increase nuclease resistance and/or serum stability or decrease immunogenicity.

The antisense polynucleotide molecule can be chemically synthesized or transcribed in vitro. The making and use of inhibitory therapeutic agents based on non-coding RNA such as ribozymes, RNAase P, siRNAs, and miRNAs are also known in the art, for example, as described in Sioud, RNA Therapeutics: Function, Design, and Delivery (Methods in Molecular Biology). Humana Press 2010.

A small RNA of the disclosure may include an antisense polynucleotide of from 10 to 100 (e.g., e.g., 11 to 99, 12, to 98, 13 to 97, 14, to 96, 15 to 95, 20 to 90, 30 to 80, 40 to 70, or to 60) nucleotides in length having complementarity sufficient to hybridize to a region within a protein-encoding mRNA transcript. For example, in some embodiments, the antisense polynucleotide may be from 11 to 99 nucleotides in length. In some embodiments, the antisense polynucleotide may be from 12 to 98 nucleotides in length. In some embodiments, the antisense polynucleotide may be from 13 to 97 nucleotides in length. In some embodiments, the antisense polynucleotide may be from 14 to 96 nucleotides in length. In some embodiments, the antisense polynucleotide may be from 15 to 95 nucleotides in length. In some embodiments, the antisense polynucleotide may be from 20 to 90 nucleotides in length. In some embodiments, the antisense polynucleotide may be from 30 to 80 nucleotides in length. In some embodiments, the antisense polynucleotide may be from 40 to nucleotides in length. In some embodiments, the antisense polynucleotide may be from 50 to 60 nucleotides in length.

In some embodiments, the antisense polynucleotide may be about 10 nucleotides in length. In some embodiments, the antisense polynucleotide may be about 11 nucleotides in length. In some embodiments, the antisense polynucleotide may be about 12 nucleotides in length. In some embodiments, the antisense polynucleotide may be about 13 nucleotides in length. In some embodiments, the antisense polynucleotide may be about 14 nucleotides in length. In some embodiments, the antisense polynucleotide may be about 15 nucleotides in length. In some embodiments, the antisense polynucleotide may be about 20 nucleotides in length. In some embodiments, the antisense polynucleotide may be about 30 nucleotides in length. In some embodiments, the antisense polynucleotide may be about 40 nucleotides in length. In some embodiments, the antisense polynucleotide may be about 50 nucleotides in length. In some embodiments, the antisense polynucleotide may be about 60 nucleotides in length. In some embodiments, the antisense polynucleotide may be about 70 nucleotides in length. In some embodiments, the antisense polynucleotide may be about 80 nucleotides in length. In some embodiments, the antisense polynucleotide may be about 90 nucleotides in length. In some embodiments, the antisense polynucleotide may be about 100 nucleotides in length.

Any suitable small RNA may be included.

In some embodiments, the antisense polynucleotide of from 10 to 100 (e.g., e.g., 11 to 99, 12, to 98, 13 to 97, 14, to 96, 15 to 95, 20 to 90, 30 to 80, 40 to 70, or 50 to 60) nucleotides in length has complementarity sufficient to hybridize to a region within a protein-encoding mRNA transcript (e.g., a human dystrophin mRNA transcript, a human fukutin mRNA transcript, a human gamma-sarcoglycan mRNA transcript, a human dysferlin mRNA transcript, a human myotonic dystrophy protein kinase mRNA transcript, a human laminin subunit alpha 2 mRNA transcript, a human usherin mRNA transcript, a human collagen alpha-1(VII) chain mRNA transcript, or a human activin A receptor type 1 mRNA transcript).

In some embodiments, the mRNA transcript is a dystrophin mRNA transcript, a fukutin mRNA transcript, a gamma-sarcoglycan mRNA transcript is a dysferlin mRNA transcript, a myotonic dystrophy protein kinase mRNA transcript, a laminin subunit alpha 2 mRNA transcript, a usherin mRNA transcript, a collagen alpha-1(VII) chain mRNA transcript, or an activin A receptor type 1 mRNA transcript.

In some embodiments, the mRNA transcript is a human dystrophin mRNA transcript, a human fukutin mRNA transcript, a human gamma-sarcoglycan mRNA transcript, a human dysferlin mRNA transcript, a human myotonic dystrophy protein kinase mRNA transcript, a human laminin subunit alpha 2 mRNA transcript, a human usherin mRNA transcript, a human collagen alpha-1(VII) chain mRNA transcript, or a human activin A receptor type 1 mRNA transcript. For example, in some embodiments, the mRNA transcript is a human dystrophin mRNA transcript. In some embodiments, the mRNA transcript is a human fukutin mRNA transcript. In some embodiments, the mRNA transcript is a human gamma-sarcoglycan mRNA transcript. In some embodiments, the mRNA transcript is a human dysferlin mRNA transcript. In some embodiments, the mRNA transcript is a human myotonic dystrophy protein kinase mRNA transcript. In some embodiments, the mRNA transcript is a human laminin subunit alpha 2 mRNA transcript. In some embodiments, the mRNA transcript is a human usherin mRNA transcript. In some embodiments, the mRNA transcript is a human collagen alpha-1(VII) chain mRNA transcript. In some embodiments, the mRNA transcript is a human activin A receptor type 1 mRNA transcript.

Any suitable mRNA transcript may be used in the compositions described herein.

### Exemplary Antisense RNAs

In one approach, the presently disclosed subject matter provides a single-stranded asRNA having a nucleobase sequence with at least 25 contiguous nucleobases complementary to an equal-length portion within the human dystrophin exon 2 target region. In some embodiments, the target region includes the region beginning at residue 17 of SEQ ID NO: 1 and ends at residue 46 of SEQ ID NO: 1 (e.g., this region includes the ESE). This approach is typically referred to as an antisense approach. Without wishing to be bound by theory, this approach involves hybridization of an oligonucleotide to a target nucleic acid (e.g., DMD exon 2 pre-mRNA, transcript 1, transcript 2, or a combination thereof), thereby sterically blocking the target nucleic acid from binding cellular post-transcription modification or translation machinery and thus preventing the function or translation of the target nucleic acid. Alternatively, and without wishing to be bound by theory, this approach involves hybridization of an oligonucleotide to a target nucleic acid (e.g., DMD exon 2 pre-mRNA, transcript 1, transcript 2, or a combination thereof), followed by ribonuclease H (RNase H) mediated cleavage of the target nucleic acid. In some embodiments, the single-stranded oligonucleotide may be delivered (e.g., to a patient) as a double stranded oligonucleotide, where the oligonucleotide is hybridized to another.

In some embodiments, the antisense polynucleotide is a DMD exon 2 ESE antisense construct, such as an asRNA, including a total of 25 to 40 (e.g., 25 to 40 (e.g., 25 to 40, 26 to 40, 27 to 40, 28 to 40, 29 to 40, 30 to 40, 31 to 40, 32 to 40, 33 to 40, 34 to 40, 35 to 40, 36 to 40, 37 to 40, 38 to 40, or 39 to 40) interlinked nucleotides and having a nucleobase sequence including at least 25 contiguous nucleobases complementary to an equal-length portion of a human DMD exon 2 target nucleic acid (e.g., residues +17 to +46 of the human DMD exon 2, relative to the nucleic acid sequence of SEQ ID NO: 1). For example, the DMD exon 2 inhibitor may include the nucleic acid sequence of SEQ ID NO: 2, or a nucleic acid sequence that includes a sequence that is at least 90% identical to the nucleic acid sequence of SEQ ID NO: 2 (e.g., 90%, 91%, 92%, 93%, 94%, 95%, 96%, 97%, 98%, 99%, or 99.9% identical to the nucleic acid sequence of SEQ ID NO: 2).

In some embodiments, the antisense polynucleotide includes a nucleobase sequence including at least 25 contiguous nucleobases complementary to a portion of residues +17 to +46 of SEQ ID NO: 1 (e.g., this includes the human dystrophin exon 2 ESE). In some embodiments, the antisense polynucleotide includes a nucleobase sequence including at least contiguous nucleobases complementary to a portion of residues +17 to +46 of SEQ ID NO: 1 (e.g., this includes the human dystrophin exon 2 ESE). In some embodiments, the antisense polynucleotide includes a nucleobase sequence including at least 27 contiguous nucleobases complementary to a portion of residues +17 to +46 of SEQ ID NO: 1 (e.g., this includes the human dystrophin exon 2 ESE). In some embodiments, the antisense polynucleotide includes a nucleobase sequence including at least 28 contiguous nucleobases complementary to a portion of residues +17 to +46 of SEQ ID NO: 1 (e.g., this includes the human dystrophin exon 2 ESE). In some embodiments, the antisense polynucleotide includes a nucleobase sequence including at least 29 contiguous nucleobases complementary to a portion of residues +17 to +46 of SEQ ID NO: 1 (e.g., this includes the human dystrophin exon 2 ESE). In some embodiments, the antisense polynucleotide includes a nucleobase sequence including at least 30 contiguous nucleobases complementary to residues +17 to +46 of SEQ ID NO: 1 (e.g., this includes the human dystrophin exon 2 ESE). In some embodiments, the antisense polynucleotide includes a nucleobase sequence including at least contiguous nucleobases complementary to residues +17 to +46 of SEQ ID NO: 1 (e.g., this includes the human dystrophin exon 2 ESE). In some embodiments, the antisense polynucleotide includes a nucleobase sequence including at least 32 contiguous nucleobases complementary to residues +17 to +46 of SEQ ID NO: 1 (e.g., this includes the human dystrophin exon 2 ESE). In some embodiments, the antisense polynucleotide includes a nucleobase sequence including at least 33 contiguous nucleobases complementary to residues +17 to +46 of SEQ ID NO: 1 (e.g., this includes the human dystrophin exon 2 ESE). In some embodiments, the antisense polynucleotide includes a nucleobase sequence including at least contiguous nucleobases complementary to residues +17 to +46 of SEQ ID NO: 1 (e.g., this includes the human dystrophin exon 2 ESE). In some embodiments, the antisense polynucleotide includes a nucleobase sequence including at least 35 contiguous nucleobases complementary to residues +17 to +46 of SEQ ID NO: 1 (e.g., this includes the human dystrophin exon 2 ESE). In some embodiments, the antisense polynucleotide includes a nucleobase sequence including at least 36 contiguous nucleobases complementary to residues +17 to +46 of SEQ ID NO: 1 (e.g., this includes the human dystrophin exon 2 ESE). In some embodiments, the antisense polynucleotide includes a nucleobase sequence including at least contiguous nucleobases complementary to residues +17 to +46 of SEQ ID NO: 1 (e.g., this includes the human dystrophin exon 2 ESE). In some embodiments, the antisense polynucleotide includes a nucleobase sequence including at least 38 contiguous nucleobases complementary to residues +17 to +46 of SEQ ID NO: 1 (e.g., this includes the human dystrophin exon 2 ESE). In some embodiments, the antisense polynucleotide includes a nucleobase sequence including at least 39 contiguous nucleobases complementary to residues +17 to +46 of SEQ ID NO: 1 (e.g., this includes the human dystrophin exon 2 ESE). In some embodiments, the antisense polynucleotide includes a nucleobase sequence including at least 40 contiguous nucleobases complementary to residues +17 to +46 of SEQ ID NO: 1 (e.g., this includes the human dystrophin exon 2 ESE).

In some embodiments, the antisense polynucleotide includes a nucleobase sequence including at least 70% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9%) complementary to the region beginning at residue 17 of SEQ ID NO: 1 and ending at residue 46 of SEQ ID NO: 1. In some embodiments, the antisense polynucleotide includes a nucleobase sequence including at least 71% complementary to the region beginning at residue 17 of SEQ ID NO: 1 and ending at residue 46 of SEQ ID NO: 1. In some embodiments, the antisense polynucleotide includes a nucleobase sequence including at least 72% complementary to the region beginning at residue 17 of SEQ ID NO: 1 and ending at residue 46 of SEQ ID NO: 1. In some embodiments, the antisense polynucleotide includes a nucleobase sequence including at least 73% complementary to the region beginning at residue 17 of SEQ ID NO: 1 and ending at residue 46 of SEQ ID NO: 1. In some embodiments, the antisense polynucleotide includes a nucleobase sequence including at least 74% complementary to the region beginning at residue 17 of SEQ ID NO: 1 and ending at residue 46 of SEQ ID NO: 1. In some embodiments, the antisense polynucleotide includes a nucleobase sequence including at least 75% complementary to the region beginning at residue 17 of SEQ ID NO: 1 and ending at residue 46 of SEQ ID NO: 1. In some embodiments, the antisense polynucleotide includes a nucleobase sequence including at least 76% complementary to the region beginning at residue 17 of SEQ ID NO: 1 and ending at residue 46 of SEQ ID NO: 1. In some embodiments, the antisense polynucleotide includes a nucleobase sequence including at least 77% complementary to the region beginning at residue 17 of SEQ ID NO: 1 and ending at residue 46 of SEQ ID NO: 1. In some embodiments, the antisense polynucleotide includes a nucleobase sequence including at least 78% complementary to the region beginning at residue 17 of SEQ ID NO: 1 and ending at residue 46 of SEQ ID NO: 1. In some embodiments, the antisense polynucleotide includes a nucleobase sequence including at least 79% complementary to the region beginning at residue 17 of SEQ ID NO: 1 and ending at residue 46 of SEQ ID NO: 1. In some embodiments, the antisense polynucleotide includes a nucleobase sequence including at least 80% complementary to the region beginning at residue 17 of SEQ ID NO: 1 and ending at residue 46 of SEQ ID NO: 1. In some embodiments, the antisense polynucleotide includes a nucleobase sequence including at least 81% complementary to the region beginning at residue 17 of SEQ ID NO: 1 and ending at residue 46 of SEQ ID NO: 1. In some embodiments, the antisense polynucleotide includes a nucleobase sequence including at least 82% complementary to the region beginning at residue 17 of SEQ ID NO: 1 and ending at residue 46 of SEQ ID NO: 1. In some embodiments, the antisense polynucleotide includes a nucleobase sequence including at least 83% complementary to the region beginning at residue 17 of SEQ ID NO: 1 and ending at residue 46 of SEQ ID NO: 1. In some embodiments, the antisense polynucleotide includes a nucleobase sequence including at least 84% complementary to the region beginning at residue 17 of SEQ ID NO: 1 and ending at residue 46 of SEQ ID NO: 1. In some embodiments, the antisense polynucleotide includes a nucleobase sequence including at least 85% complementary to the region beginning at residue 17 of SEQ ID NO: 1 and ending at residue 46 of SEQ ID NO: 1. In some embodiments, the antisense polynucleotide includes a nucleobase sequence including at least 86% complementary to the region beginning at residue 17 of SEQ ID NO: 1 and ending at residue 46 of SEQ ID NO: 1. In some embodiments, the antisense polynucleotide includes a nucleobase sequence including at least 87% complementary to the region beginning at residue 17 of SEQ ID NO: 1 and ending at residue 46 of SEQ ID NO: 1. In some embodiments, the antisense polynucleotide includes a nucleobase sequence including at least 88% complementary to the region beginning at residue 17 of SEQ ID NO: 1 and ending at residue 46 of SEQ ID NO: 1. In some embodiments, the antisense polynucleotide includes a nucleobase sequence including at least 89% complementary to the region beginning at residue 17 of SEQ ID NO: 1 and ending at residue 46 of SEQ ID NO: 1. In some embodiments, the antisense polynucleotide includes a nucleobase sequence including at least 90% complementary to the region beginning at residue 17 of SEQ ID NO: 1 and ending at residue 46 of SEQ ID NO: 1. In some embodiments, the antisense polynucleotide includes a nucleobase sequence including at least 91% complementary to the region beginning at residue 17 of SEQ ID NO: 1 and ending at residue 46 of SEQ ID NO: 1. In some embodiments, the antisense polynucleotide includes a nucleobase sequence including at least 92% complementary to the region beginning at residue 17 of SEQ ID NO: 1 and ending at residue 46 of SEQ ID NO: 1. In some embodiments, the antisense polynucleotide includes a nucleobase sequence including at least 93% complementary to the region beginning at residue 17 of SEQ ID NO: 1 and ending at residue 46 of SEQ ID NO: 1. In some embodiments, the antisense polynucleotide includes a nucleobase sequence including at least 94% complementary to the region beginning at residue 17 of SEQ ID NO: 1 and ending at residue 46 of SEQ ID NO: 1. In some embodiments, the antisense polynucleotide includes a nucleobase sequence including at least 95% complementary to the region beginning at residue 17 of SEQ ID NO: 1 and ending at residue 46 of SEQ ID NO: 1. In some embodiments, the antisense polynucleotide includes a nucleobase sequence including at least 96% complementary to the region beginning at residue 17 of SEQ ID NO: 1 and ending at residue 46 of SEQ ID NO: 1. In some embodiments, the antisense polynucleotide includes a nucleobase sequence including at least 97% complementary to the region beginning at residue 17 of SEQ ID NO: 1 and ending at residue 46 of SEQ ID NO: 1. In some embodiments, the antisense polynucleotide includes a nucleobase sequence including at least 98% complementary to the region beginning at residue 17 of SEQ ID NO: 1 and ending at residue 46 of SEQ ID NO: 1. In some embodiments, the antisense polynucleotide includes a nucleobase sequence including at least 99% complementary to the region beginning at residue 17 of SEQ ID NO: 1 and ending at residue 46 of SEQ ID NO: 1.

In some embodiments, the antisense polynucleotide includes a nucleobase sequence that is fully complementary to the region beginning at residue 17 of SEQ ID NO: 1 and ending at residue 46 of SEQ ID NO: 1. In some embodiments, the nucleobase sequence of the antisense polynucleotide that is fully complementary includes at least 10 (e.g., at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30) contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 11 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 12 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 13 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 14 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 15 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 16 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 17 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 18 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 19 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 20 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 21 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 22 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 23 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 24 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 25 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 26 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 27 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 28 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 29 contiguous nucleotides that are fully complementary to a contiguous polynucleotide segment of equal length within the region beginning at residue 17 of SEQ ID NO: 1 and ending at residue 46 of SEQ ID NO: 1. In some embodiments, the antisense polynucleotide includes at least 30 contiguous nucleotides that are fully complementary to a contiguous polynucleotide segment of equal length within the region beginning at residue 17 of SEQ ID NO: 1 and ending at residue 46 of SEQ ID NO: 1.

In some embodiments, the antisense polynucleotide includes from 10 to 30 (e.g., 11 to 30, 12 to 30, 13 to 30, 14 to 30, 15 to 30, 16 to 30, 17 to 30, 18 to 30, 19 to 30, 20 to 30, 21 to 30, 22 to 30, 23 to 30, 24 to 30, 25 to 30, 26 to 30, 27 to 30, 28 to 30, or 29 to 30) contiguous nucleotides that are fully complementary to a contiguous polynucleotide segment of equal length within the region beginning at residue 17 of SEQ ID NO: 1 and ending at residue 46 of SEQ ID NO: 1. For example, the antisense polynucleotide includes 10 contiguous nucleotides that are fully complementary. For example, the antisense polynucleotide includes contiguous nucleotides that are fully complementary. For example, the antisense polynucleotide includes 12 contiguous nucleotides that are fully complementary. For example, the antisense polynucleotide includes 13 contiguous nucleotides that are fully complementary. For example, the antisense polynucleotide includes 14 contiguous nucleotides that are fully complementary. For example, the antisense polynucleotide includes contiguous nucleotides that are fully complementary. For example, the antisense polynucleotide includes 16 contiguous nucleotides that are fully complementary. For example, the antisense polynucleotide includes 17 contiguous nucleotides that are fully complementary. For example, the antisense polynucleotide includes 18 contiguous nucleotides that are fully complementary. For example, the antisense polynucleotide includes contiguous nucleotides that are fully complementary. For example, the antisense polynucleotide includes 20 contiguous nucleotides that are fully complementary. For example, the antisense polynucleotide includes 21 contiguous nucleotides that are fully complementary. For example, the antisense polynucleotide includes 22 contiguous nucleotides that are fully complementary. For example, the antisense polynucleotide includes contiguous nucleotides that are fully complementary. For example, the antisense polynucleotide includes 24 contiguous nucleotides that are fully complementary. For example, the antisense polynucleotide includes 25 contiguous nucleotides that are fully complementary. For example, the antisense polynucleotide includes 26 contiguous nucleotides that are fully complementary. For example, the antisense polynucleotide includes contiguous nucleotides that are fully complementary. For example, the antisense polynucleotide includes 28 contiguous nucleotides that are fully complementary. For example, the antisense polynucleotide includes 29 contiguous nucleotides that are fully complementary. For example, the antisense polynucleotide includes 30 contiguous nucleotides that are fully complementary.

In some embodiments, the antisense polynucleotide includes 9 or fewer (e.g., 8, 7, 6, 5, 4, 3, 2, or 1) nucleotide mismatches relative to the region beginning at residue 17 of SEQ ID NO: 1 and ending at residue 46 of SEQ ID NO: 1. For example, the antisense polynucleotide includes 8 nucleotide mismatches relative to the region beginning at residue of SEQ ID NO: 1 and ending at residue 46 of SEQ ID NO: 1. For example, the antisense polynucleotide includes 7 nucleotide mismatches relative to the region beginning at residue of SEQ ID NO: 1 and ending at residue 46 of SEQ ID NO: 1. For example, the antisense polynucleotide includes 6 nucleotide mismatches relative to the region beginning at residue of SEQ ID NO: 1 and ending at residue 46 of SEQ ID NO: 1. For example, the antisense polynucleotide includes 5 nucleotide mismatches relative to the region beginning at residue of SEQ ID NO: 1 and ending at residue 46 of SEQ ID NO: 1. For example, the antisense polynucleotide includes 4 nucleotide mismatches relative to the region beginning at residue 17 of SEQ ID NO: 1 and ending at residue 46 of SEQ ID NO: 1. For example, the antisense polynucleotide includes 3 nucleotide mismatches relative to the region beginning at residue of SEQ ID NO: 1 and ending at residue 46 of SEQ ID NO: 1. For example, the antisense polynucleotide includes 2 nucleotide mismatches relative to the region beginning at residue of SEQ ID NO: 1 and ending at residue 46 of SEQ ID NO: 1. For example, the antisense polynucleotide includes 1 nucleotide mismatches relative to the region beginning at residue of SEQ ID NO: 1 and ending at residue 46 of SEQ ID NO: 1.

In some embodiments, the antisense polynucleotide has a nucleic acid sequence that is at least 85% (e.g., 86%, 87%, 88%, 89%, 90%, 91%, 92%, 93%, 94%, 95%, 96%, 97%, 98%, 99%, or 99.9%) identical to the nucleic acid sequence of SEQ ID NO: 2. As described herein, SEQ ID NO: 2 refers to the nucleic acid sequence: UUUACCCAUUUUGUGAAUGUUUUCUUUUGA. In some embodiments, the antisense polynucleotide has a nucleic acid sequence that is 86% identical to the nucleic acid sequence of SEQ ID NO: 2. In some embodiments, the antisense polynucleotide has a nucleic acid sequence that is 87% identical to the nucleic acid sequence of SEQ ID NO: 2. In some embodiments, the antisense polynucleotide has a nucleic acid sequence that is 88% identical to the nucleic acid sequence of SEQ ID NO: 2. In some embodiments, the antisense polynucleotide has a nucleic acid sequence that is 89% identical to the nucleic acid sequence of SEQ ID NO: 2. In some embodiments, the antisense polynucleotide has a nucleic acid sequence that is 90% identical to the nucleic acid sequence of SEQ ID NO: 2. In some embodiments, the antisense polynucleotide has a nucleic acid sequence that is 91% identical to the nucleic acid sequence of SEQ ID NO: 2. In some embodiments, the antisense polynucleotide has a nucleic acid sequence that is 92% identical to the nucleic acid sequence of SEQ ID NO: 2. In some embodiments, the antisense polynucleotide has a nucleic acid sequence that is 93% identical to the nucleic acid sequence of SEQ ID NO: 2. In some embodiments, the antisense polynucleotide has a nucleic acid sequence that is 94% identical to the nucleic acid sequence of SEQ ID NO: 2. In some embodiments, the antisense polynucleotide has a nucleic acid sequence that is 95% identical to the nucleic acid sequence of SEQ ID NO: 2. In some embodiments, the antisense polynucleotide has a nucleic acid sequence that is 96% identical to the nucleic acid sequence of SEQ ID NO: 2. In some embodiments, the antisense polynucleotide has a nucleic acid sequence that is 97% identical to the nucleic acid sequence of SEQ ID NO: 2. In some embodiments, the antisense polynucleotide has a nucleic acid sequence that is 98% identical to the nucleic acid sequence of SEQ ID NO: 2. In some embodiments, the antisense polynucleotide has a nucleic acid sequence that is 99% identical to the nucleic acid sequence of SEQ ID NO: 2. In some embodiments, the antisense polynucleotide has a nucleic acid sequence that is identical to the nucleic acid sequence of SEQ ID NO: 2.

In some embodiments, the antisense polynucleotide does not have complementarity to exon 2 of a human dystrophin RNA transcript at a site located 5′ to residue 17 of SEQ ID NO: 1.

In some embodiments, the antisense polynucleotide does not have complementarity to exon 2 of a human dystrophin RNA transcript at a site located 3′ to residue 46 of SEQ ID NO: 1.

### U7 Small Nuclear Ribonucleic Acids

In some embodiments, the disclosure includes U7 snRNAs for the delivery of an antisense polynucleotide (e.g., an asRNA, a siRNA, a shRNA, a miRNA, or an ASO). snRNAs are a class of small RNA molecules that are found within the splicing speckles and Cajal bodies of the cell nucleus in eukaryotic cells. snRNAs are associated with a set of specific proteins, and the complexes are referred to as small nuclear ribonucleoproteins (snRNP). Each snRNP particle is composed of a snRNA component and several snRNP-specific proteins (e.g., Sm proteins). The snRNAs, along with their associated proteins (e.g., Sm proteins), form ribonucleoprotein complexes (snRNPs), which bind to specific sequences on the pre-mRNA substrate. They are transcribed by either RNA polymerase II or RNA polymerase III. snRNAs are often divided into two classes based upon both common sequence features and associated protein factors, such as the RNA-binding LSm proteins. The first class, known as Sm-class snRNA, consists of U1, U2, U4, U4atac, U5, U7, U11, and U12, which are transcribed by RNA polymerase II. The second class, known as Lsm-class snRNA, consists of U6 and U6atac, which are transcribed by RNA polymerase III and remain in the nucleus, in contrast to Sm-class snRNA.

In some embodiments, the disclosure uses U7 snRNA molecules to deliver antisense polynucleotides that mask the binding or the utilization of an ESE or one or more splice sites (e.g., SA, SD, or a combination thereof) during the splicing reaction. snRNA is normally involved in histone pre-mRNA 3′ end processing but, in some aspects, is converted into a versatile tool for splicing modulation or as asRNA that is continuously expressed in cells e.g., see Goyenvalle et al., Science 306(5702): 1796-9 (2004). By replacing the wild-type U7 Sm binding site with a consensus sequence derived from spliceosomal snRNAs, the resulting RNA assembles with the seven Sm proteins found in spliceosomal snRNAs. As a result, modified (e.g., optimized) U7 Sm (U7 Sm OPT) RNA accumulates more efficiently in the nucleoplasm and will no longer mediate histone pre-mRNA cleavage, although it can still bind to histone pre-mRNA and act as a competitive inhibitor for wild-type U7 snRNPs. By further replacing the sequence binding to the histone downstream element with one complementary to a particular target in a splicing substrate, it is possible to create U7 snRNAs capable of modulating specific splicing events. The advantage of using U7 derivatives is that the antisense sequence is embedded into a snRNP complex. Moreover, when embedded into a gene therapy vector, these small RNAs can be permanently expressed inside the target cell after a single injection (e.g., see Levy et al., Eur. J. Hum. Genet. 18(9): 969-70 (2010); Wein et al., Hum. Mutat. 31(2): 136-42, (2010); Wein et al., Nat. Med. 20(9): 992-1000 (2014)).

In some embodiments, the U7 snRNA (e.g., for the delivery of an antisense polynucleotide) is located 3′ to an antisense polynucleotide.

In some embodiments, the U7 snRNA includes an Sm OPT motif. In some embodiments, the Sm OPT motif has a nucleic acid sequence that is at least 85% (e.g., 86%, 87%, 88%, 89%, 90%, 91%, 92%, 93%, 94%, 95%, 96%, 97%, 98%, 99%, or 99.9%) identical to the nucleic acid sequence of SEQ ID NO: 5. In some embodiments, the Sm OPT motif has a nucleic acid sequence that is 86% identical to the nucleic acid sequence of SEQ ID NO: 5. In some embodiments, the Sm OPT motif has a nucleic acid sequence that is 87% identical to the nucleic acid sequence of SEQ ID NO: 5. In some embodiments, the Sm OPT motif has a nucleic acid sequence that is 88% identical to the nucleic acid sequence of SEQ ID NO: 5. In some embodiments, the Sm OPT motif has a nucleic acid sequence that is 89% identical to the nucleic acid sequence of SEQ ID NO: 5. In some embodiments, the Sm OPT motif has a nucleic acid sequence that is 90% identical to the nucleic acid sequence of SEQ ID NO: 5. In some embodiments, the Sm OPT motif has a nucleic acid sequence that is 91% identical to the nucleic acid sequence of SEQ ID NO: 5. In some embodiments, the Sm OPT motif has a nucleic acid sequence that is 92% identical to the nucleic acid sequence of SEQ ID NO: 5. In some embodiments, the Sm OPT motif has a nucleic acid sequence that is 93% identical to the nucleic acid sequence of SEQ ID NO: 5. In some embodiments, the Sm OPT motif has a nucleic acid sequence that is 94% identical to the nucleic acid sequence of SEQ ID NO: 5. In some embodiments, the Sm OPT motif has a nucleic acid sequence that is 95% identical to the nucleic acid sequence of SEQ ID NO: 5. In some embodiments, the Sm OPT motif has a nucleic acid sequence that is 96% identical to the nucleic acid sequence of SEQ ID NO: 5. In some embodiments, the Sm OPT motif has a nucleic acid sequence that is 97% identical to the nucleic acid sequence of SEQ ID NO: 5. In some embodiments, the Sm OPT motif has a nucleic acid sequence that is 98% identical to the nucleic acid sequence of SEQ ID NO: 5. In some embodiments, the Sm OPT motif has a nucleic acid sequence that is 99% identical to the nucleic acid sequence of SEQ ID NO: 5. In some embodiments, the Sm OPT motif has a nucleic acid sequence that is identical to the nucleic acid sequence of SEQ ID NO: 5.

In some embodiments, the U7 snRNA includes a U7 stem loop. In some embodiments, the U7 stem loop has a nucleic acid sequence that is at least 85% (e.g., 86%, 87%, 88%, 89%, 90%, 91%, 92%, 93%, 94%, 95%, 96%, 97%, 98%, 99%, or 99.9%) identical to the nucleic acid sequence of SEQ ID NO: 6. In some embodiments, the U7 stem loop has a nucleic acid sequence that is 86% identical to the nucleic acid sequence of SEQ ID NO: 6. In some embodiments, the U7 stem loop has a nucleic acid sequence that is 87% identical to the nucleic acid sequence of SEQ ID NO: 6. In some embodiments, the U7 stem loop has a nucleic acid sequence that is 88% identical to the nucleic acid sequence of SEQ ID NO: 6. In some embodiments, the U7 stem loop has a nucleic acid sequence that is 89% identical to the nucleic acid sequence of SEQ ID NO: 6. In some embodiments, the U7 stem loop has a nucleic acid sequence that is 90% identical to the nucleic acid sequence of SEQ ID NO: 6. In some embodiments, the U7 stem loop has a nucleic acid sequence that is 91% identical to the nucleic acid sequence of SEQ ID NO: 6. In some embodiments, the U7 stem loop has a nucleic acid sequence that is 92% identical to the nucleic acid sequence of SEQ ID NO: 6. In some embodiments, the U7 stem loop has a nucleic acid sequence that is 93% identical to the nucleic acid sequence of SEQ ID NO: 6. In some embodiments, the U7 stem loop has a nucleic acid sequence that is 94% identical to the nucleic acid sequence of SEQ ID NO: 6. In some embodiments, the U7 stem loop has a nucleic acid sequence that is 95% identical to the nucleic acid sequence of SEQ ID NO: 6. In some embodiments, the U7 stem loop has a nucleic acid sequence that is 96% identical to the nucleic acid sequence of SEQ ID NO: 6. In some embodiments, the U7 stem loop has a nucleic acid sequence that is 97% identical to the nucleic acid sequence of SEQ ID NO: 6. In some embodiments, the U7 stem loop has a nucleic acid sequence that is 98% identical to the nucleic acid sequence of SEQ ID NO: 6. In some embodiments, the U7 stem loop has a nucleic acid sequence that is 99% identical to the nucleic acid sequence of SEQ ID NO: 6. In some embodiments, the U7 stem loop has a nucleic acid sequence that is identical to the nucleic acid sequence of SEQ ID NO: 6.

In some embodiments, the U7 snRNA includes a U7 downstream region. In some embodiments, the U7 downstream region has a nucleic acid sequence that is at least 85% (e.g., 86%, 87%, 88%, 89%, 90%, 91%, 92%, 93%, 94%, 95%, 96%, 97%, 98%, 99%, or 99.9%) identical to the nucleic acid sequence of SEQ ID NO: 7. In some embodiments, the U7 downstream region has a nucleic acid sequence that is 86% identical to the nucleic acid sequence of SEQ ID NO: 7. In some embodiments, the U7 downstream region has a nucleic acid sequence that is 87% identical to the nucleic acid sequence of SEQ ID NO: 7. In some embodiments, the U7 downstream region has a nucleic acid sequence that is 88% identical to the nucleic acid sequence of SEQ ID NO: 7. In some embodiments, the U7 downstream region has a nucleic acid sequence that is 89% identical to the nucleic acid sequence of SEQ ID NO: 7. In some embodiments, the U7 downstream region has a nucleic acid sequence that is 90% identical to the nucleic acid sequence of SEQ ID NO: 7. In some embodiments, the U7 downstream region has a nucleic acid sequence that is 91% identical to the nucleic acid sequence of SEQ ID NO: 7. In some embodiments, the U7 downstream region has a nucleic acid sequence that is 92% identical to the nucleic acid sequence of SEQ ID NO: 7. In some embodiments, the U7 downstream region has a nucleic acid sequence that is 93% identical to the nucleic acid sequence of SEQ ID NO: 7. In some embodiments, the U7 downstream region has a nucleic acid sequence that is 94% identical to the nucleic acid sequence of SEQ ID NO: 7. In some embodiments, the U7 downstream region has a nucleic acid sequence that is 95% identical to the nucleic acid sequence of SEQ ID NO: 7. In some embodiments, the U7 downstream region has a nucleic acid sequence that is 96% identical to the nucleic acid sequence of SEQ ID NO: 7. In some embodiments, the U7 downstream region has a nucleic acid sequence that is 97% identical to the nucleic acid sequence of SEQ ID NO: 7. In some embodiments, the U7 downstream region has a nucleic acid sequence that is 98% identical to the nucleic acid sequence of SEQ ID NO: 7. In some embodiments, the U7 downstream region has a nucleic acid sequence that is 99% identical to the nucleic acid sequence of SEQ ID NO: 7. In some embodiments, the U7 downstream region has a nucleic acid sequence that is identical to the nucleic acid sequence of SEQ ID NO: 7.

In some embodiments, the U7 snRNA (e.g., including or encoding sequences of an Sm OPT, U7 stem loop, and U7 downstream region) has a nucleic acid sequence that is at least 85% (e.g., 86%, 87%, 88%, 89%, 90%, 91%, 92%, 93%, 94%, 95%, 96%, 97%, 98%, 99%, or 99.9%) identical to the nucleic acid sequence of SEQ ID NO: 8. In some embodiments, the U7 snRNA has a nucleic acid sequence that is 86% identical to the nucleic acid sequence of SEQ ID NO: 8. In some embodiments, the U7 snRNA has a nucleic acid sequence that is 87% identical to the nucleic acid sequence of SEQ ID NO: 8. In some embodiments, the U7 snRNA has a nucleic acid sequence that is 88% identical to the nucleic acid sequence of SEQ ID NO: 8. In some embodiments, the U7 snRNA has a nucleic acid sequence that is 89% identical to the nucleic acid sequence of SEQ ID NO: 8. In some embodiments, the U7 snRNA has a nucleic acid sequence that is 90% identical to the nucleic acid sequence of SEQ ID NO: 8. In some embodiments, the U7 snRNA has a nucleic acid sequence that is 91% identical to the nucleic acid sequence of SEQ ID NO: 8. In some embodiments, the U7 snRNA has a nucleic acid sequence that is 92% identical to the nucleic acid sequence of SEQ ID NO: 8. In some embodiments, the U7 snRNA has a nucleic acid sequence that is 93% identical to the nucleic acid sequence of SEQ ID NO: 8. In some embodiments, the U7 snRNA has a nucleic acid sequence that is 94% identical to the nucleic acid sequence of SEQ ID NO: 8. In some embodiments, the U7 snRNA has a nucleic acid sequence that is 95% identical to the nucleic acid sequence of SEQ ID NO: 8. In some embodiments, the U7 snRNA has a nucleic acid sequence that is 96% identical to the nucleic acid sequence of SEQ ID NO: 8. In some embodiments, the U7 snRNA has a nucleic acid sequence that is 97% identical to the nucleic acid sequence of SEQ ID NO: 8. In some embodiments, the U7 snRNA has a nucleic acid sequence that is 98% identical to the nucleic acid sequence of SEQ ID NO: 8. In some embodiments, the U7 snRNA has a nucleic acid sequence that is 99% identical to the nucleic acid sequence of SEQ ID NO: 8. In some embodiments, the U7 snRNA has a nucleic acid sequence that is identical to the nucleic acid sequence of SEQ ID NO: 8, shown below:

AAUUUUUGGAGCAGGUUUUCUGACUUCGGUCGGAAAACCCCUCCC
AAUUUCACUGGUCUACAAUGAAAGCAAAACAGUUCUCUUCCCCGC
UCCCCGGUGUGUGAGAGGGGCUUUGAUCCUUCUCUGGUUUCCUAG
GAAACGCGUAUGUG.

As described herein, exemplary U7 snRNA elements, including a BoxB RNA element, an asRNA, asRNA tail, Sm OPT motif, a U7 stem loop, a U7 downstream region, and a U7 snRNA, as descried herein, are exemplified by the nucleic acid sequences in Table 1, shown below.

TABLE 1
U7 snRNA Nucleic Acid Sequences

| SEQ | | |
|---|---|---|
| ID | | Genetic |
| NO: | Nucleic Acid Sequence | Component |
| 3 | GCCCUGAAGAGGGC | BoxB RNA |
| | | element |
| 2 | UUUACCCAUUUUGUGAAUGUUUUCUUUUGA | asRNA |
| 5 | AAUUUUUGGAG | Sm OPT |
| | | motif |
| 6 | CAGGUUUUCUGACUUCGGUCGGAAAACCCCU | U7 stem |
| | | loop |
| 7 | CCCAAUUUCACUGGUCUACAAUGAAAGCAAA | U7 |
| | ACAGUUCUCUUCCCCGCUCCCCGGUGUGUGA | downstream |
| | GAGGGGCUUUGAUCCUUCUCUGGUUUCCUAG | region |
| | GAAACGCGUAUGUG | |
| 8 | AAUUUUUGGAGCAGGUUUUCUGACUUCGGUC | U7 snRNA |
| | GGAAAACCCCUCCCAAUUUCACUGGUCUACA | |
| | AUGAAAGCAAAACAGUUCUCUUCCCCGCUCC | |
| | CCGGUGUGUGAGAGGGGCUUUGAUCCUUCUC | |
| | UGGUUUCCUAGGAAACGCGUAUGUG | |

In some embodiments, a U7 expression cassette, described herein, is exemplified by the nucleic acid sequence of SEQ ID NO: 9, shown below:

TAACAACATAGGAGCTGTGATTGGCTGTTTTCAGCCAATCAGCAC
TGACTCATTTGCATAGCCTTTACAAGCGGTCACAAACTCAAGAAA
CGAGCGGTTTTAATAGTCTTTTAGAATATTGTTTATCGAACCGAA
TAAGGAACTGTGCTTTGTGATTCACATATCAGTGGAGGGGTGTGG
AAATGGCACCTTGATCTCACCCTCATCGAAAGTGGAGTTGATGTC
CTTCCCTGGCTCGCTACAGACGCACTTCCGCAAAGAGGGCCCTGA
AGAGGGCCCTTTTCTTATGATAGGGACTTAGGGTGTTTACCCATT
TTGTGAATGTTTTCTTTTGAAATTTTTGGAGCAGGTTTTCTGACT
TCGGTCGGAAAACCCCTCCCAATTTCACTGGTCTACAATGAAAGC
AAAACAGTTCTCTTCCCCGCTCCCCGGTGTGTGAGAGGGGCTTTG
ATCCTTCTCTGGTTTCCTAGGAAACGCGTATGTG.

### Delivery

### I. Viral Vectors for Expression of Therapeutic Transgenes

Viral genomes provide a rich source of vectors that can be used for the efficient delivery of exogenous genes into a mammalian cell (e.g., a muscle cell or a neuron). Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996)). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

### IA. Retroviral Vectors

The delivery vector used in the methods and compositions described herein may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the transgene. An overview of optimization strategies for packaging and transducing LVs is provided in Delenda, J Gene Med. 6: S125 (2004), the disclosure of which is incorporated herein by reference.

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral complimentary DNA (cDNA) deprived of all open reading frames, but maintaining the sequences required for replication, encapsidation, and expression, in which the sequences to be expressed are inserted.

A LV used in the methods and compositions described herein may include one or more of a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR′ backbone, which may include for example as provided below.

The Lentigen LV described in Lu et al., J. Gene Med. 6:963 (2004) may be used to express the DNA molecules and/or transduce cells. A LV used in the methods and compositions described herein may a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating L TR (SIN-LTR). It will be readily apparent to one skilled in the art that optionally one or more of these regions is substituted with another region performing a similar function.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a WPRE. The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of transgene expression from several different promoters, both in vitro and in vivo. The LV used in the methods and compositions described herein may include both a cPPT sequence and WPRE sequence. The vector may also include an IRES sequence that permits the expression of multiple polypeptides from a single promoter.

In addition to IRES sequences, other elements which permit expression of multiple polypeptides are useful. The vector used in the methods and compositions described herein may include multiple promoters that permit expression more than one polypeptide. The vector used in the methods and compositions described herein may include a protein cleavage site that allows expression of more than one polypeptide. Examples of protein cleavage sites that allow expression of more than one polypeptide are described in Klump et al., Gene Ther. 8:811 (2001), Osborn et al., Mol. Ther. 12:569 (2005), Szymczak and Vignali, Expert Opin Biol Ther. 5:627 (2005), and Szymczak et al., Nat Biotechnol. 22:589 (2004), the disclosures of which are incorporated herein by reference as they pertain to protein cleavage sites that allow expression of more than one polypeptide. It will be readily apparent to one skilled in the art that other elements that permit expression of multiple polypeptides identified in the future are useful and may be utilized in the vectors suitable for use with the compositions and methods described herein.

The vector used in the methods and compositions described herein may be a clinical grade vector.

### IB. Adeno-Associated Viral Vectors

Nucleic acids of the compositions and methods described herein may be incorporated into a recombinant linear adeno-associated virus (rAAV) vector, a recombinant self-complementary AAV (scAAV) vector, and/or virions, in order to facilitate their introduction into a cell (e.g., a muscle cell or a neuron). Adeno-associated virus (AAV) vectors can be used in the central nervous system, and appropriate promoters and serotypes are discussed in Pignataro et al., J Neural Transm., 125: 575 (2018), the disclosure of which is incorporated herein by reference as it pertains to promoters and AAV serotypes useful in CNS gene therapy. In some embodiments, the AAV is a single-stranded rAAV. In some embodiments, the AAV is a scAAV.

rAAV vectors useful in the compositions and methods described herein are recombinant nucleic acid constructs (e.g., nucleic acids capable of expression in muscle cells or neurons) that include (1) a heterologous sequence to be expressed and (2) viral sequences that facilitate integration and expression of the heterologous genes. The viral sequences may include those sequences of AAV that are required in cis for replication and packaging (e.g., functional inverted terminal repeat sequences (ITR)) of the DNA into a virion. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. In some embodiments, the AAV ITR is an AAV2 ITR. Methods for using rAAV vectors are described, for example, in Tai et al., J Biomed. Sci. 7:279 (2000), and Monahan and Samulski, Gene Delivery 7:24 (2000), the disclosures of each of which are incorporated herein by reference as they pertain to AAV vectors for gene delivery.

Enhancer elements can be used to increase expression of modified DNA molecules. In some embodiments, an enhancer element that promotes expression of the antisense polynucleotide in a muscle cell or neuron is used in conjunction with the compositions and methods of the disclosure. Exemplary enhancers that may be used in conjunction with the compositions and methods of the disclosure are a muscle creatine kinase (MCK) enhancer, a desmin enhancer, a myosin light chain enhancer, a myosin heavy chain enhancer, a cardiac troponin C enhancer, a troponin I enhancer, a myoD gene family enhancer, an actin alpha enhancer, an actin beta enhancer, an actin gamma enhancer, or an enhancer within intron 1 of ocular paired like homeodomain 3. In some embodiments, the enhancer is an MCK enhancer.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2, and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, rh10 and rh74. For targeting cells located in or delivered to the central nervous system, AAV2, AAV9, and AAV10 may be particularly useful. Construction and use of AAV vectors and AAV proteins of different serotypes are described, for example, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV3, AAV4, AAV5, AAV6, AAV7, AAV8, etc.). For example, a representative pseudotyped vector is an AAV8

vector encoding a therapeutic protein pseudotyped with a capsid gene derived from AAV serotype 2. Techniques involving the construction and use of pseudotyped rAAV virions are known in the art and are described, for example, in Duan et al., J. Virol. 75:7662-7671 (2001); Halbert et al., J. Virol. 74:1524-1532 (2000); Zolotukhin et al., Methods, 28:158-167 (2002); and Auricchio et al., Hum. Molec. Genet., 10:3075-3081 (2001); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

In some embodiments, the transgene (e.g., including or encoding a BoxB RNA element and an antisense polynucleotide) is operably linked to a U7 promoter. In some embodiments, the 5′ end of the antisense polynucleotide (e.g., including or encoding an interfering RNA) is bound to the 3′ end of the U7 promoter. In some embodiments, the U7 promoter has a nucleic acid sequence that is at least 85% (e.g., 86%, 87%, 88%, 89%, 90%, 91%, 92%, 93%, 94%, 95%, 96%, 97%, 98%, 99%, or 99.9%) identical to the nucleic acid sequence of SEQ ID NO: 4. In some embodiments, the U7 promoter has a nucleic acid sequence that is 86% identical to the nucleic acid sequence of SEQ ID NO: 4. In some embodiments, the U7 promoter has a nucleic acid sequence that is 87% identical to the nucleic acid sequence of SEQ ID NO: 4. In some embodiments, the U7 promoter has a nucleic acid sequence that is 88% identical to the nucleic acid sequence of SEQ ID NO: 4. In some embodiments, the U7 promoter has a nucleic acid sequence that is 89% identical to the nucleic acid sequence of SEQ ID NO: 4. In some embodiments, the U7 promoter has a nucleic acid sequence that is 90% identical to the nucleic acid sequence of SEQ ID NO: 4. In some embodiments, the U7 promoter has a nucleic acid sequence that is 91% identical to the nucleic acid sequence of SEQ ID NO: 4. In some embodiments, the U7 promoter has a nucleic acid sequence that is 92% identical to the nucleic acid sequence of SEQ ID NO: 4. In some embodiments, the U7 promoter has a nucleic acid sequence that is 93% identical to the nucleic acid sequence of SEQ ID NO: 4. In some embodiments, the U7 promoter has a nucleic acid sequence that is 94% identical to the nucleic acid sequence of SEQ ID NO: 4. In some embodiments, the U7 promoter has a nucleic acid sequence that is 95% identical to the nucleic acid sequence of SEQ ID NO: 4. In some embodiments, the U7 promoter has a nucleic acid sequence that is 96% identical to the nucleic acid sequence of SEQ ID NO: 4. In some embodiments, the U7 promoter has a nucleic acid sequence that is 97% identical to the nucleic acid sequence of SEQ ID NO: 4. In some embodiments, the U7 promoter has a nucleic acid sequence that is 98% identical to the nucleic acid sequence of SEQ ID NO: 4. In some embodiments, the U7 promoter has a nucleic acid sequence that is 99% identical to the nucleic acid sequence of SEQ ID NO: 4. In some embodiments, the U7 promoter has a nucleic acid sequence that is identical to the nucleic acid sequence of SEQ ID NO: 4.

In some embodiments, the MCK enhancer has a nucleic acid sequence that is at least 85% (e.g., 86%, 87%, 88%, 89%, 90%, 91%, 92%, 93%, 94%, 95%, 96%, 97%, 98%, 99%, or 99.9%) identical to the nucleic acid sequence of SEQ ID NO: 11. In some embodiments the MCK enhancer has a nucleic acid sequence that is 86% identical to the nucleic acid sequence of SEQ ID NO: 11. In some embodiments, the MCK enhancer has a nucleic acid sequence that is 87% identical to the nucleic acid sequence of SEQ ID NO: 11. In some embodiments, the MCK enhancer has a nucleic acid sequence that is 88% identical to the nucleic acid sequence of SEQ ID NO: 11. In some embodiments, the MCK enhancer has a nucleic acid sequence that is 89% identical to the nucleic acid sequence of SEQ ID NO: 11. In some embodiments, the MCK enhancer has a nucleic acid sequence that is 90% identical to the nucleic acid sequence of SEQ ID NO: 11. In some embodiments, the MCK enhancer has a nucleic acid sequence that is 91% identical to the nucleic acid sequence of SEQ ID NO: 11. In some embodiments, the MCK enhancer has a nucleic acid sequence that is 92% identical to the nucleic acid sequence of SEQ ID NO: 11. In some embodiments, the MCK enhancer has a nucleic acid sequence that is 93% identical to the nucleic acid sequence of SEQ ID NO: 11. In some embodiments, the MCK enhancer has a nucleic acid sequence that is 94% identical to the nucleic acid sequence of SEQ ID NO: 11. In some embodiments, the MCK enhancer has a nucleic acid sequence that is 95% identical to the nucleic acid sequence of SEQ ID NO: 11. In some embodiments, the MCK enhancer has a nucleic acid sequence that is 96% identical to the nucleic acid sequence of SEQ ID NO: 11. In some embodiments, the MCK enhancer has a nucleic acid sequence that is 97% identical to the nucleic acid sequence of SEQ ID NO: 11. In some embodiments, the MCK enhancer has a nucleic acid sequence that is 98% identical to the nucleic acid sequence of SEQ ID NO: 11. In some embodiments, the MCK enhancer has a nucleic acid sequence that is 99% identical to the nucleic acid sequence of SEQ ID NO: 11. In some embodiments, the MCK enhancer has a nucleic acid sequence that is identical to the nucleic acid sequence of SEQ ID NO: 11.

In some embodiments, the AAV encodes a transgene that has a nucleic acid sequence that is at least 85% (e.g., 86%, 87%, 88%, 89%, 90%, 91%, 92%, 93%, 94%, 95%, 96%, 97%, 98%, 99%, or 99.9%) identical to the nucleic acid sequence of SEQ ID NO: 9. In some embodiments, the AAV encodes a transgene that has a nucleic acid sequence that is at least 86% identical to the nucleic acid sequence of SEQ ID NO: 9. In some embodiments, the AAV encodes a transgene that has a nucleic acid sequence that is at least 87% identical to the nucleic acid sequence of SEQ ID NO: 9. In some embodiments, the AAV encodes a transgene that has a nucleic acid sequence that is at least 88% identical to the nucleic acid sequence of SEQ ID NO: 9. In some embodiments, the AAV encodes a transgene that has a nucleic acid sequence that is at least 89% identical to the nucleic acid sequence of SEQ ID NO: 9. In some embodiments, the AAV encodes a transgene that has a nucleic acid sequence that is at least 90% identical to the nucleic acid sequence of SEQ ID NO: 9. In some embodiments, the AAV encodes a transgene that has a nucleic acid sequence that is at least 91% identical to the nucleic acid sequence of SEQ ID NO: 9. In some embodiments, the AAV encodes a transgene that has a nucleic acid sequence that is at least 92% identical to the nucleic acid sequence of SEQ ID NO: 9. In some embodiments, the AAV encodes a transgene that has a nucleic acid sequence that is at least 93% identical to the nucleic acid sequence of SEQ ID NO: 9. In some embodiments, the AAV encodes a transgene that has a nucleic acid sequence that is at least 94% identical to the nucleic acid sequence of SEQ ID NO: 9. In some embodiments, the AAV encodes a transgene that has a nucleic acid sequence that is at least 95% identical to the nucleic acid sequence of SEQ ID NO: 9. In some embodiments, the AAV encodes a transgene that has a nucleic acid sequence that is at least 96% identical to the nucleic acid sequence of SEQ ID NO: 9. In some embodiments, the AAV encodes a transgene that has a nucleic acid sequence that is at least 97% identical to the nucleic acid sequence of SEQ ID NO: 9. In some embodiments, the AAV encodes a transgene that has a nucleic acid sequence that is at least 98% identical to the nucleic acid sequence of SEQ ID NO: 9. In some embodiments, the AAV encodes a transgene that has a nucleic acid sequence that is at least 99% identical to the nucleic acid sequence of SEQ ID NO: 9. In some embodiments, the AAV encodes a transgene that has a nucleic acid sequence that is identical to the nucleic acid sequence of SEQ ID NO: 9.

In some embodiments, the AAV has a nucleic acid sequence that is at least 85% (e.g., 86%, 87%, 88%, 89%, 90%, 91%, 92%, 93%, 94%, 95%, 96%, 97%, 98%, 99%, or 99.9%) identical to the nucleic acid sequence of SEQ ID NO: 10. In some embodiments, the AAV has a nucleic acid sequence that is 86% identical to the nucleic acid sequence of SEQ ID NO: 10. In some embodiments, the AAV has a nucleic acid sequence that is 87% identical to the nucleic acid sequence of SEQ ID NO: 10. In some embodiments, the AAV has a nucleic acid sequence that is 88% identical to the nucleic acid sequence of SEQ ID NO: 10. In some embodiments, the AAV has a nucleic acid sequence that is 89% identical to the nucleic acid sequence of SEQ ID NO: 10. In some embodiments, the AAV has a nucleic acid sequence that is 90% identical to the nucleic acid sequence of SEQ ID NO: 10. In some embodiments, the AAV has a nucleic acid sequence that is 91% identical to the nucleic acid sequence of SEQ ID NO: 10. In some embodiments, the AAV has a nucleic acid sequence that is 92% identical to the nucleic acid sequence of SEQ ID NO: 10. In some embodiments, the AAV has a nucleic acid sequence that is 93% identical to the nucleic acid sequence of SEQ ID NO: 10. In some embodiments, the AAV has a nucleic acid sequence that is 94% identical to the nucleic acid sequence of SEQ ID NO: 10. In some embodiments, the AAV has a nucleic acid sequence that is 95% identical to the nucleic acid sequence of SEQ ID NO: 10. In some embodiments, the AAV has a nucleic acid sequence that is 96% identical to the nucleic acid sequence of SEQ ID NO: 10. In some embodiments, the AAV has a nucleic acid sequence that is 97% identical to the nucleic acid sequence of SEQ ID NO: 10. In some embodiments, the AAV has a nucleic acid sequence that is 98% identical to the nucleic acid sequence of SEQ ID NO: 10. In some embodiments, the AAV has a nucleic acid sequence that is 99% identical to the nucleic acid sequence of SEQ ID NO: 10. In some embodiments, the AAV has a nucleic acid sequence that is identical to the nucleic acid sequence of SEQ ID NO: 10. As described herein, exemplary AAV vector components, including an U7 promoter and MCK enhancer, as described herein, are exemplified by the nucleic acid sequences in Table 2, shown below.

TABLE 2
Exemplary AAV Vector Components
Nucleic Acid Sequences

| SEQ ID | | Genetic |
|---|---|---|
| NO: | Nucleic Acid Sequence | Component |
| 4 | TAACAACATAGGAGCTGTGATTGGC | U7 |
| | TGTTTTCAGCCAATCAGCACTGACT | promoter |
| | CATTTGCATAGCCTTTACAAGCGGT | |
| | CACAAACTCAAGAAACGAGCGGTTT | |
| | TAATAGTCTTTTAGAATATTGTTTA | |
| | TCGAACCGAATAAGGAACTGTGCTT | |
| | TGTGATTCACATATCAGTGGAGGGG | |
| | TGTGGAAATGGCACCTTGATCTCAC | |
| | CCTCATCGAAAGTGGAGTTGATGTC | |
| | CTTCCCTGGCTCGCTACAGACGCAC | |
| | TTCCGCA | |
| 11 | CCACTACGGGTCTAGGCTGCCCATG | MCK |
| | TAAGGAGGCAAGGCCTGGGGACACC | enhancer |
| | CGAGATGCCTGGTTATAATTAACCC | |
| | AGACATGTGGCTGCCCCCCCCCCCC | |
| | CAACACCTGCTGCCTGAGCCTCACC | |
| | CCCACCCCGGTGCCTGGGTCTTAGG | |
| | CTCTGTACACCATGGAGGAGAAGCT | |
| | CGCTCTAAAAATAACCCTGTCCCTG | |
| | GTGGATC | |

As described herein, an exemplary AAV2/8 vector having the nucleic acid sequence of SEQ ID NO: 10, is shown below:

AGCGCGCAAAGCCACTACTGCCACTTTTGGAGACTGTGTACGTCG
AGGGCCTGCAGTAACAGCTAGGACGTCCGCATGCGGTAGGTTACC
TTTTCCCCCCCAACCTAAAGCGAACAACGTATCCAACCAGAGTTT
GAGGACCGGAGTTCACTAAGAGGACGGAGACGGAGGGTTTCACGA
CTCTAATGTCCACACTCCGTGGTACGGTCCAGAGAATGACAAACA
TTAATTTATGTATGTGTAAAACACACAAACACACGTGGAAATATT
TCAGTTTCCACTATCATTGGGTAAATTCAAGGATGAGTTAAAATG
AAAGGTCCCTATTGATTGATGAAAAAGAAAAACTCTACCTCAGAG
CGACACATCGGGTCCGACCTCACGTCACCGTGGTAGAGCCGAGTG
ACGTTCGAGGAGGAGGGACCAAGTGCGATAAGAGGACGGAGTCGG
AGGGGTTGTTGATCCTGATGTCCGAGTGGAGCGGTATGGACCGAT
TAAAAAACATAAAAATCATCTCTGTCCCAAAGTGACACAATCGGT
CCTACCAGAGCTAGAGGACTGGAACACTAGGCGGACGGAGACGGA
GGGTTTCACGACCCTAATGTCCGTACTCGTTGGAGTGGGTCGACC
CTATTGATGAAAAATGTCCAACTATAAGAAAACCTGAAAAGGGGA
CACATTTTTATATGATATAAACAATACATGTATAATACATGTATG
TCTGTGTTTAACCTGGTAAGAGTCATATTACTAAGAGTCCAAAAA
AAAAAAAAAAAACTCCACCCCTTGATCTATTAATACCTGTAGAAA
GGTATGATCGTATAGTTATAGATGGAGTAAGAAAAATTATAAAAA
CGATCATAAGGTAACATACTTACAGGATACTAAATGAATTGGACA
GGTAGTTATAAACAAAGGTCCAAAAACGATAATATTACGACGACG
TTTCATGTAGGAGTGTGTAGAAATAAAACAGATAAGTATAAAGAC
ATTCTATCCAATGATTTCAACCTTGACGGTTTAATTGTGATAGTA
TGATAAAACAAAAAATTAAAATTAAAAAATTTTTTACATTTTACA
CGTTAAAGTTCTCCTCTTTGAACTTGTGTTCCTCGTTTTAGATAA
AAATATTGTAGGATAATTTTCGAACGAAATGTATTTCTAAAACTT
TCTTATCGTATTTATGTTCTAAAGATAAAATTAACCTAAGAATCC
CGATTATTTTATTAGTCGGAATCGTGAATAAATAAATAAAAAAAA
CTCTCCCTCAGAGCGAGACAACAGGTACGACCTCACGTCACCGCA
CTAGAGCCGAGTGACGTTCGAGGTGGAGTACTCAAGTGTGGTAAG
AGGACGGAGTCAGAGGGCTCATCGACCCTGAGGTCCGCGGGAGAT
GTTTCGGGCAGATTAAAAAAAACATAAAAATCATCTCTGTCCCAA
AGTGACACAATCGGTCCTACCAGAACTAGAGGACTGGAACACTAG
ACGGGCGGAGCCGGAGGGTTTCACGACCCTAATATCCGAACTCGG
TGACGAGGGCCGGTCGTGAATAAAAATATTAAGAAGTACTAATGA
CACAATGACAGGGTACCCGGCGGTCCCGGTCGATCCAACCGGTGA
GGGAGAGACGCGCGAGCGAGCGAGTGACTCCGGCCCGCTGGTTTC
CAGCGGGCTGCGGGCCCGAAACGGGCCCGCCGGAGTCACTCGCTC
GCTCGCGCGTCTCTCCCTCACCGGTTGAGGTAGTGATCCCCAAGG
AGGATCGTGCGCATCTTAGTCTTTTAGACTCTTCTTTTGAATTTT
TGGAAACTTAGATTGGATGAGTTTATTTGAAACTTATATAAATAA
CTTTATAAATTAAACAAATAAAAAATAAAAAAATAAAATCTCTGT
TCCAGAGTGATTTCACGTCCGATCTCACGTCACTGTGTTAGTACC
GATACCGAGTGACGTTGGAGTTTGAGGACCCGAGTTCGCTAAGAC
AACGGAATCCAAGCGGTCCTCGACCGTGATGTCCACGGTGGTGTG
GACCGAAAAAACAAAACAAAAAAAACCCATCTCTTCCCCTAAACC
ATACAACGAATCCGACTAGAACTTGATCGGAGTGCGTTAGGACGA
AGCCGAAAGGTTTTACAACCCTAATATCCGTACTCGGTACGCGGA
CTGGAACTAATGGAGCTACTACACATACAGTATGTAACCTCCCGT
TTCTGTAGAGACTTAAGGAGTGTTGTCGGTACCGTCCCCCGTCCA
TGGGTGTATGCGCAAAGGATCCTTTGGTCTCTTCCTAGTTTCGGG
GAGAGTGTGTGGCCCCTCGCCCCTTCTCTTGACAAAACGAAAGTA
ACATCTGGTCACTTTAACCCTCCCCAAAAGGCTGGCTTCAGTCTT
TTGGACGAGGTTTTTAAAGTTTTCTTTTGTAAGTGTTTTACCCAT
TTGTGGGATTCAGGGATAGTATTCTTTTCCCGGGAGAAGTCCCGG
GAGAAACGCCTTCACGCAGACATCGCTCGGTCCCTTCCTGTAGTT
GAGGTGAAAGCTACTCCCACTCTAGTTCCACGGTAAAGGTGTGGG
GAGGTGACTATACACTTAGTGTTTCGTGTCAAGGAATAAGCCAAG
CTATTTGTTATAAGATTTTCTGATAATTTTGGCGAGCAAAGAACT
CAAACACTGGCGAACATTTCCGATACGTTTACTCAGTCACGACTA
ACCGACTTTTGTCGGTTAGTGTCGAGGATACAACAATCTAGGTGG
TCCCTGTCCCAATAAAAATCTCGCTCGAAGAGGAGGTACCACATG
TCTCGGATTCTGGGTCCGTGGCCCCACCCCCACTCCGAGTCCGTC
GTCCACAACCCCCCCCCCCCCGTCGGTGTACAGACCCAATTAATA
TTGGTCCGTAGAGCCCACAGGGGTCCGGAACGGAGGAATGTACCC
GTCGGATCTGGGCATCACCCCATGGGTACAATGTCTACTGTCTGA
CTACGTATCTCTTCAATTCAGCACCCCTCAAATGAAAGAGGATTT
AACAGGACAATGATCTACTTAAACAAAAACAAAGTAAAACAAAAC
AAAAACTCTGTCTCAGAGTACAACAGTGGGTCTGACCTCACGTCA
CCGAGGTAGAGCCGAGTGATATCGGAGGCGGAGGACTCAAGTTCA
CTAAGAGGACGGAGTCGGAGGGTTCATCGACCCACGTACGGTGGT
GCGGACCGATTAAAAACATAAAAATCATATCTACCCCAAAGTGGT
ACAACCGGTCTGACCAGAGCTTGAGGACTGGAGTTCACTAGGTGG
GGCGCGAATCGGAGGGTTTCACGACCCTAATATCCGCACTCGGTG
GTGTGGTCCGGTACTTAAACAAAAGTTATAAATAAATAAAACATA
AAAGATAAAAACTCTACCTCAGAGCGAGACGACAGGTCCGATCTC
ACGTCACCACACTAGAACCGAGTGACATCGGAGGTGGAGGACTCA
AGTTCACTAAGAGGACGGAATCGGAGGGCTCATCGACCCTAATGT
CCGCGGGTGGTAGTGTGGGCCGATTAAAAACATAAAAATCATCTC
TACCCCAAAGTGGTACAACCGGTGCGACCAGAGTTTGACTGGAGT
TAACTAGGTGGGTGGAACTGGAGGGTTTCACGACCCTAATGTCTG
GACTCGGTGTCGCGGGTCGGGAAGTTATAAATAAATTTAAACGGA
CGACCGATTGAAGAGTAACGTGGACCCGAGATCACATTAATTTAA
TGAAGTAAGAGAAAAATTTTGAAAAATGAAAAAAGAAAAAACACA
AAAAGTAAGAGAATAGATGCTCTCGGTGTTATGAACTTCTGTGGT
TAACTATGGGGAATCAGTGTAGACTCGATTTGTGAAAGTCAAGGA
TGTCGACAAAGAATTAGAATCCAGTGTACCAAAGAAGGGTACGAC
AAGAAGGGTCTGTCGTAAAAAAAAAAAAAACTCTCAGAGTGAGAC
AACGGGTCCGACCTCATGTCATCGTGTTAGAGTCAAATGACGTTG
GAGACGGAAGGTCCAAGTCCACTAAGAGGACGAAGTCGGAGGACT
CATCGACCCTGATGTCCTCGCACGGTGGTGCGGGCCGATTAAAAA
CATAAAAATCATCTCTGTCCCAAAGTGGTACAACCGGTCCGACCA
GAGCTTGAGGAATGGAACACTAGGCGGACAGAGCCGGAGGGTTTC
ACCACCCTAATGTCCACACTCGGTGGTGCGGACCAAGAATGTAAA
TAAAACCTTATTTAAATCTATGTGTCTTTTCAACGTTTCTATTCT
CAAAGGTATATTGGGAGTGGGTCAAACGGAAGGGATTACAATTGT
AGAATGTAATAGTACCATGTAAACAGTTTTGATTCTGTGAAAAAA
AAGATTATTTTTATTATCTCTACTCCAGAGTGATATAACAGGTCC
GACCAGAGTTTGAAACTCGAGTTCGTCAGGAGGGTGGAGGTGGAG
GGTTTCACGACCCTAATGTCCGTACTTGGTGGTGTGGGTCGGATG
TAACAATACAATGATGAGAGGTCTGATAAGTCTAAAGTGGTTAAA
AAGATAGGTATCGGAAAAAAAGACAAAGTCCTAGGGGTGATGCCC
AGATCCGACGGGTACATTCCTCCGTTCCGGACCCCTGTGGGCTCT
ACGGACCAATATTAATTGGGTCTGTACACCGACGGGGGGGGGGGG
GTTGTGGACGACGGACTCGGAGTGGGGGTGGGGCCACGGACCCAG
AATCCGAGACATGTGGTACCTCCTCTTCGAGCGAGATTTTTATTG
GGACAGGGACCACCTAGATTGTTGTATCCTCGACACTAACCGACA
AAAGTCGGTTAGTCGTGACTGAGTAAACGTATCGGAAATGTTCGC
CAGTGTTTGAGTTCTTTGCTCGCCAAAATTATCAGAAAATCTTAT
AACAAATAGCTTGGCTTATTCCTTGACACGAAACACTAAGTGTAT
AGTCACCTCCCCACACCTTTACCGTGGAACTAGAGTGGGAGTAGC
TTTCACCTCAACTACAGGAAGGGACCGAGCGATGTCTGCGTGAAG
GCGTTTCTCCCGGGACTTCTCCCGGGAAAAGAATACTATCCCTGA
ATCCCACAAATGGGTAAAACACTTACAAAAGAAAACTTTAAAAAC
CTCGTCCAAAAGACTGAAGCCAGCCTTTTGGGGAGGGTTAAAGTG
ACCAGATGTTACTTTCGTTTTGTCAAGAGAAGGGGCGAGGGGCCA
CACACTCTCCCCGAAACTAGGAAGAGACCAAAGGATCCTTTGCGC
ATACACCCTAGGTTAAGTCCTGTGGTATGGTGACATACCAATTAA
AATTTTTGAGTTTACAACCCTCAAACGAAATAGTGGCGACATAGA
ATAGTAGAGCAATCTATTAAAACATAGACCTCGGACGAGAGATCG
TACTTATTCATTTTTACACCTGAAACACTAAATGTCTAGACTATA
AATACAAAAACACAAAAATGAAATTCATGATCTGTTTCATTTAGA
TTCTCCAGACACCATGACGTGATCTCTTAACAATGAACTAATACC
ACCCGGTTCACCAACTTTTAAATGGATTGTTATGATATTGTATCC
GGTATAAAGTATTAAAATTAGTGTTCCGTTAACACTTTTAAAATA
GTTTACAAATTATTTTCGTTCCACTTCTTCCACTATCGAAATTTA
AATGAACGGTAAAAACCCGTGTGTTTTCATTAAACGAGACGGTGA
ATCTCAATATTCCAGTTTCACCCTCATTTATTAGAAACATAATCC
TTACGCCGGCGAGATCCTCCTTGGGGATCACTACCTCAACCGGTG
AGGGAGAGACGCGCGAGCGAGCGAGTGACTCCGGCGGGCCCGTTT
CGGGCCCGCAGCCCGCTGGAAACCAGCGGGCCGGAGTCACTCGCT
CGCTCGCGCGTCTCTCCCTCACCGGTTGGATCTCCGGCGGTCCCG
GTATAAAGAGTTAAAAATTTAAAAAGTTTTTTTAATTAGGAATTA
CACGTATAAAAACTTAACAATTATATTGAAAAACTCCACTACAGA
AGTACACAAAGTTGATGAATTTTTGAAAATTTGTCATATATTATT
TTTTAGAAGGTCCGGTGAGTGTGGACATTAGGGTCGTGAAACCCT
CCGACTCCACCCGTCTAGTGGACTCCCGTCCTCAAGCTCTGGTCG
GACCGGTTATATATATATAAGTATATAAGTATATATATATATATA
AGTATATAAGTATATATATATAAGTATATAAGTATATATATATAT
ATATATATATATCGTTTTGGAGTAGAGATTATTTTATGTTTTTAA
TCGACTCGCACCACTACCTACGGACATCAGGGTCGATGAGCCCTC
CGACTCCGTCCTCTTAGAGAACTTGGACCCTCCACCTCCAACGTC
ACTCGACTCTACCACGGTGACGGGAGGTCGGACTCACTGTCTCGC
TCTGAGCCAGAGGTTTTTTTTTGTTGTTTTTTTAGAAGGTAGGAA
CAGAGGGTAGGTGGGGAAGGGGGGTCGTACATGAACGTCTGAAAT
ACGTATATGTCACTCATGACATATATGTGTTTATTATTTTTTTAG
TATATATATTATATACATTAAGGGGAAATGTACTTTCCATCGTGT
GACCAGACATGTCAGACAGACGTGACACGATAAAGTGAAATATAA
AAATATCAAACTGTCTCAAGATTGTAAAGAAAAAAAAAAAAAAAT
TGTCTCAGAACAAGGACTAACAATTTAAAATTTCGTAGGATTTCA
AACCAAAGTGTGAACTTACTTATGGTACATTCCTAAGTGAATGTA
TCTACACCAACGGACTTAGAATTCTTATTTTATTGTAACAAACAT
AAATAAATTTAATCACAAGGAAAATACCAAACGGACTTTCGTGTT
GTTTTAGGAGTGGTTCTATAATGTTAATACTGAGGGTATGTCCAT
TTGACAAATCTCTAACCGTTCGTGGAAAATTACTTTCCTCAGTCG
GTCGAATCACACGTCATAAATAAAGACGGCCTTCTCCCTCGAAGT
CCCTGTCTGAAACCAAATCAGTACTTCGGAGGTCGTGAGGGTTCG
CCAACACCAACTGGTTCGTTAAATACGAAAATGGAAAGATGAAGG
TCTCCGAACAAATGAATAGTCATTCGTAATTAAATCACAGGGGAG
TCTACGGAAAATGAAAGAAGAAAAGACGGATCTTATTCGACGAGA
AGGTTAAAACGTCGATGTACAAAGGTGGGGTCAACCTTAAAGAGG
TATTGTAGGTAACATCGATAGGAAGTTAGATGTCGGAGATAAAGG
ACAATATCGACCAGTCCAGATTAGGGAGTTTTATGAGACAGGGGA
CGAAGGGAATAGACGACCGGTGGAAAAAGGGGGTGTATGTGTGAC
GGTACAGGGTGGGAAGTGAGTTCAACAAGGGACGGTGGAGTTGTT
TAAATTCAGGTATTTTGGTAGGTTACCCGAGCTCGGGACGTCCTA
GTAACAGTGTACACTCGTTTTCCGGTCGTTTTCCGGTCCTTGGCA
TTTTTCCGGCGCAACGACCGCAAAAAGGTATCCGAGGGGGGGGAC
TGCTCGTAGTGTTTTTAGCTGCGAGTTCAGTCTCCACCGCTTTGG
GCTGTCCTGATATTTCTATGGTCCGCAAAGGGGGACCTTCGAGGG
AGCACGCGAGAGGACAAGGCTGGGACGGCGAATGGCCTATGGACA
GGCGGAAAGAGGGAAGCCCTTCGCACCGCGAAAGAGTATCGAGTG
CGACATCCATAGAGTCAAGCCACATCCAGCAAGCGAGGTTCGACC
CGACACACGTGCTTGGGGGGCAAGTCGGGCTGGCGACGCGGAATA
GGCCATTGATAGCAGAACTCAGGTTGGGCCATTCTGTGCTGAATA
GCGGTGACCGTCGTCGGTGACCATTGTCCTAATCGTCTCGCTCCA
TACATCCGCCACGATGTCTCAAGAACTTCACCACCGGATTGATGC
CGATGTGATCTTCTTGTCATAAACCATAGACGCGAGACGACTTCG
GTCAATGGAAGCCTTTTTCTCAACCATCGAGAACTAGGCCGTTTG
TTTGGTGGCGACCATCGCCACCAAAAAAACAAACGTTCGTCGTCT
AATGCGCGTCTTTTTTTCCTAGAGTTCTTCTAGGAAACTAGAAAA
GATGCCCCAGACTGCGAGTCACCTTGCTTTTGAGTGCAATTCCCT
AAAACCAGTACTCTAATAGTTTTTCCTAGAAGTGGATCTAGGAAA
ATTTAATTTTTACTTCAAAATTTAGTTCGGGTTAGACTTATTACA
ATGTTGGTTAATTGGTTAAGACTAATCTTTTTGAGTAGCTCGTAG
TTTACTTTGACGTTAAATAAGTATAGTCCTAATAGTTATGGTATA
AAAACTTTTTCGGCAAAGACATTACTTCCTCTTTTGAGTGGCTCC
GTCAAGGTATCCTACCGTTCTAGGACCATAGCCAGACGCTAAGGC
TGAGCAGGTTGTAGTTATGTTGGATAATTAAAGGGGAGCAGTTTT
TATTCCAATAGTTCACTCTTTAGTGGTACTCACTGCTGACTTAGG
CCACTCTTACCGTTTTCAAATACGTAAAGAAAGGTCTGAACAAGT
TGTCCGGTCGGTAATGCGAGCAGTAGTTTTAGTGAGCGTAGTTGG
TTTGGCAATAAGTAAGCACTAACGCGGACTCGCTCTGCTTTATGC
GCTAGCGACAATTTTCCTGTTAATGTTTGTCCTTAGCTTACGTTG
GCCGCGTCCTTGTGACGGTCGCGTAGTTGTTATAAAAGTGGACTT
AGTCCTATAAGAAGATTATGGACCTTACGACAAAAAGGCCCCTAG
CGTCACCACTCATTGGTACGTAGTAGTCCTCATGCCTATTTTACG
AACTACCAGCCTTCTCCGTATTTAAGGCAGTCGGTCAAATCAGAC
TGGTAGAGTAGACATTGTAGTAACCGTTGCGATGGAAACGGTACA
AAGTCTTTGTTGAGACCGCGTAGCCCGAAGGGTATGTTCGCTATC
TAACAGCGTGGACTAACGGGCTGTAATAGCGCTCGGGTAAATATG
GGTATATTTAGTCGTAGGTACAACCTTAAATTAGCGCCGGAGCTG
CAAAGGGCAACTTATACCGAGTATTGTGGGGAACATAATGACAAA
TACATTCGTCTGTCAAAATAACAAGTACTACTATATAAAAATAGA
ACACGTTACATTGTAGTCTCTAAAACTCTGTGCCCGGTCTCGACG
T

### II. Methods for the Delivery of Exogenous Nucleic Acids to Target Cells

Techniques that can be used to introduce a polynucleotide, such as DNA or RNA (e.g., mRNA, transfer RNA, asRNA, siRNA, shRNA, miRNA, ASO, or chemically modified RNA), including codon-optimized DNA or RNA, into a mammalian cell (e.g., a muscle cell or a neuron) are well known in the art. For example, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids (e.g., nucleic acids capable of expression in e.g., muscle cells or neurons). Electroporation of mammalian cells is described in detail, e.g., in Chu et al., Nucleic Acids Res 15:1311 (1987), the disclosure of which is incorporated herein by reference. A similar technique, NUCLEOFECTION™, utilizes an applied electric field in order to stimulate the uptake of exogenous polynucleotides into the nucleus of a eukaryotic cell. NUCLEOFECTION™ and protocols useful for performing this technique are described in detail, e.g., in Distler et al., Exp. Dermatol. 14:315 (2005), as well as in US 2010/0317114, the disclosures of each of which are incorporated herein by reference.

An additional technique useful for the transfection of target cells is the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., JoVE 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids are contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Top Curr Chem 228:227 (2003), the disclosure of which is incorporated herein by reference) polyethylenimine, and DEAE-dextran, the use of which as a transfection agent is described in detail, for example, in Gulick et al., Curr Protoc Mol Biol 40:1:9.2:9.2.1 (1997), the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107:25 1870 (2010), the disclosure of which is incorporated herein by reference.

MAGNETOFECTION™ can also be used to deliver nucleic acids to target cells. The principle of MAGNETOFECTION™ is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, asRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Ther. 9:102 (2002), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for example, in US2010/0227406, the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane permeabilize the cells and allow polynucleotides to penetrate the cell membrane. This technique is described in detail, e.g., in Rhodes et al., Methods Cell Biol. 82:309 (2007), the disclosure of which is incorporated herein by reference.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Pharmaceutical Compositions

The RNA molecules described herein can be formulated into pharmaceutical compositions for administration to a patient, such as a human patient exhibiting or at risk of a muscular dystrophy (e.g., DMD), in a biologically compatible form suitable for administration in vivo. A pharmaceutical composition containing, for example, one or more transgenes, each encoding a BoxB RNA element and an antisense polynucleotide described herein, typically includes a pharmaceutically acceptable diluent or carrier. A pharmaceutical composition may include (e.g., consist of), e.g., a sterile saline solution and a nucleic acid. The sterile saline is typically a pharmaceutical grade saline. A pharmaceutical composition may include (e.g., consist of), e.g., sterile water and a nucleic acid. The sterile water is typically a pharmaceutical grade water. A pharmaceutical composition may include (e.g., consist of), e.g., phosphate-buffered saline (PBS) and a nucleic acid. The sterile PBS is typically a pharmaceutical grade PBS.

In certain embodiments, pharmaceutical compositions include one or more RNA molecules and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, RNA molecules may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions including a RNA molecule encompass any pharmaceutically acceptable salts of the inhibitor, esters of the inhibitor, or salts of such esters. In certain embodiments, pharmaceutical compositions including a RNA molecule, upon administration to a subject (e.g., a human), are capable of providing (directly or indirectly) the biologically active metabolite or residue thereof. Accordingly, for example, the disclosure is also drawn to pharmaceutically acceptable salts of inhibitors, prodrugs, pharmaceutically acceptable salts of such prodrugs, and other bioequivalents. Suitable pharmaceutically acceptable salts include, but are not limited to, sodium and potassium salts. In certain embodiments, prodrugs include one or more conjugate group attached to a RNA molecule, wherein the conjugate group is cleaved by endogenous nucleases within the body.

Lipid moieties have been used in nucleic acid therapies in a variety of methods. In certain such methods, the nucleic acid is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions include a delivery system. Examples of delivery systems include, but are not limited to, liposomes and emulsions. Certain delivery systems are useful for preparing certain pharmaceutical compositions including those including hydrophobic compounds. In certain embodiments, certain organic solvents such as dimethylsulfoxide are used.

In certain embodiments, pharmaceutical compositions include one or more tissue-specific delivery molecules designed to deliver the one or more pharmaceutical agents of the presently disclosed subject matter to specific tissues or cell types. For example, in certain embodiments, pharmaceutical compositions include liposomes coated with a tissue-specific antibody.

In certain embodiments, pharmaceutical compositions include a co-solvent system. Certain of such co-solvent systems include, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol including 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intraocular (e.g., intravitreal), intravenous, subcutaneous, intramuscular, intrathecal, intracerebroventricular, etc.). In certain of such embodiments, a pharmaceutical composition includes a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

### Kits

The compositions described herein can be provided in a kit for use in treating a muscular dystrophy (e.g., DMD). The kit may include one or more RNA molecules as described herein. In some embodiments, the kit may include a viral vector as described herein. In some embodiments, the kit may include a pharmaceutical composition as described herein.

The kit can include a package insert that instructs a user of the kit, such as a physician, to perform any one of the methods described herein. In some embodiments, the kit can include a package insert instructing a user of the kit to administer the composition or vector to a human patient diagnosed as having a frameshift mutation. In some embodiments, the kit can include a package insert instructing a user of the kit to administer the composition or vector to a human patient diagnosed as having overexpression of a protein of interest (e.g., dystrophin). In some embodiments, the kit can include a package insert instructing a user of the kit to administer the composition or vector to a human patient diagnosed as having DMD (e.g., the patient may be diagnosed as having a duplication in exon 2 of an endogenous DMD gene or the patient may be diagnosed as having a frameshift mutation in any one of exons 1-4 of an endogenous DMD gene).

The kit may optionally include a syringe or other device for administering the composition. In some embodiments, the kit may include one or more additional therapeutic agents.

### Combination Therapies

A transgene or RNA molecule described herein can be administered in combination with a one or more additional therapeutic agents for treatment of a muscular dystrophy (e.g., DMD). The one or more additional therapeutic agents may include a corticosteroid (e.g., bethamethasone, prednisolone, triamcinolone, methylprednisolone, dexamethasone, hydrocortisone, cortisone, ethamethasoneb, prednisone, prednisolone, triamcinolone, dexamethasone, or fludrocortisone) or an immunosuppressive drug (e.g., pomalidomide, methotrexate, azathioprine, lenalidomide, azathioprine, or thalidomide), or a combination thereof.

### EXAMPLES

The following examples are put forth so as to provide those of ordinary skill in the art with a description of how the compositions and methods described herein may be used and evaluated and are intended to be purely exemplary of the presently disclosed subject matter and are not intended to limit the scope of what the inventors regard as their presently disclosed subject matter.

### Example 1. Methods and Materials

### DMD Model

As previously described, an immortalized cell line expressing a conditionally inducible myogenic (MyoD) fibroblasts (fibroMyoD) under the control of a tetracycline-inducible promoter was developed, e.g., see U.S. Pat. No. 9,862,945. This was achieved by stable transduction of the primary fibroblast lines with a lentivirus encoding a tetracycline-inducible MyoD and containing the human telomerase gene. The resultant stable line allows MyoD expression to be initiated by treatment with doxycycline. Said cell line was generated from patients with DMD who carry a duplication of dystrophin (DMD) exon 2.

U7 snRNA Constructs

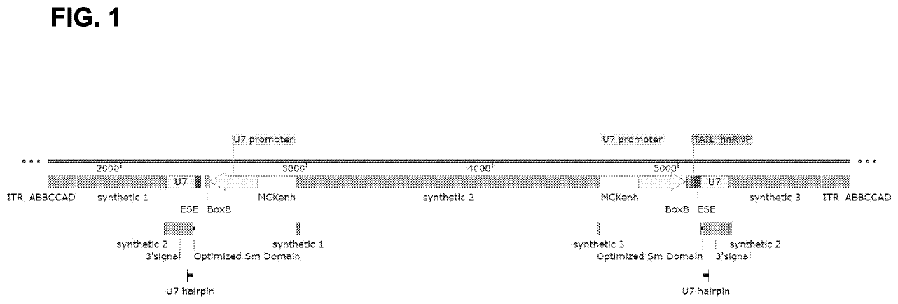

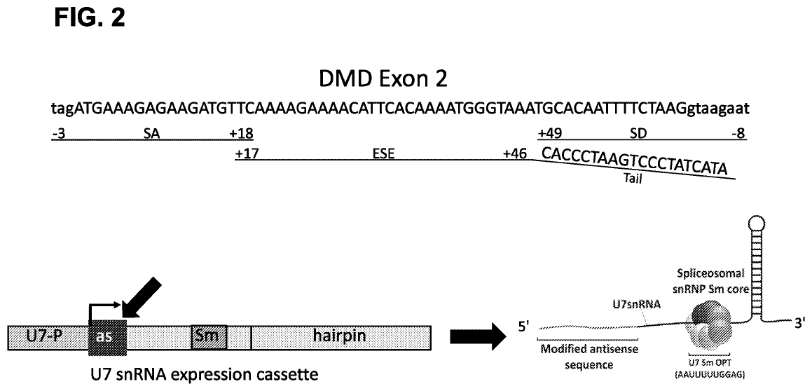

Products and methods for virally-mediated exon skipping of duplicated DMD exon 2 were developed (FIGS. 1 and 2). The products and methods were modified compared to the U7 snRNA systems described in Goyenvalle et al., Science, 306(5702): 1796-1799 (2004) and Goyenvalle et al., Mol. Ther., 20(6): 179601799 (2004), whilst the further addition of a BoxB RNA element was modified compared to U.S. Patent Application No. 63/191,069. Specifically, U7 snRNA expression cassettes (e.g., a cassette including, from 5′ to 3′, a BoxB RNA element, an antisense polynucleotide (e.g., an antisense RNA (asRNA) sequence), a U7 snRNA sequence, a U7 Sm binding protein (U7 Sm OPT) sequence, a U7 stem loop, and a U7 downstream element) were modified to include (i) a BoxB RNA element and (ii) an asRNA complementary to a region beginning at residue 17 and ending at residue 46 (SEQ ID NO: 2) of the DMD exon 2 target gene (e.g., SEQ ID NO: 1) in order to interfere with the splicing of DMD exon 2 (FIG. 2).

Exemplary U7 snRNA constructs include two BoxB RNA elements and two asRNA complementary to the exon 2 target sequence (including the ESE), oriented bi-directionally.

Respective U7 snRNA expression cassettes were cloned into an AAV plasmid with genomes including one or more of the U7 snRNA constructs.

Using a Dup2 immortalized human fibromyoblast line and the U7 snRNA expression cassettes encoding a BoxB RNA element and an asRNA described herein, DMD exon 2 duplication skipping was demonstrated, as described in Example 2.

### Example 2. Effectiveness of U7 snRNA-Mediated Skipping on Exon 2 Duplication Mutations

This Example describes the efficacy of U7 snRNA-mediated skipping on DMD Exon 2, for example, with a BoxB RNA element-containing U7 snRNA in an immortalized fibroMyoD cell line, for the amelioration of DMD mutations related to exon 2 duplication.

### Materials and Methods

Materials and Methods are described in Example 1.

### Results

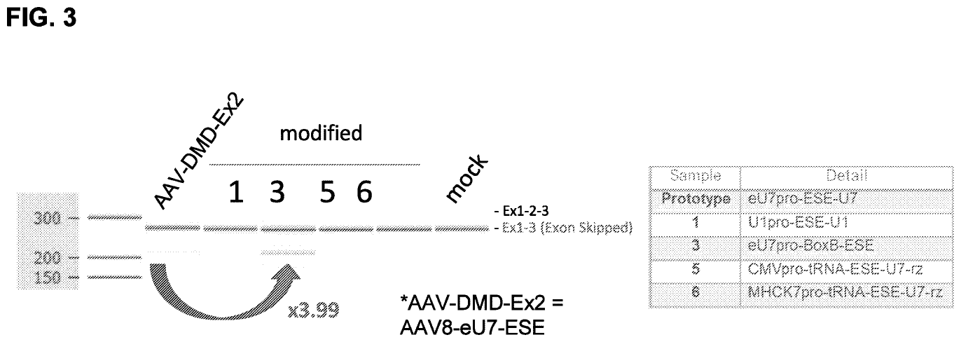

FIG. 3 shows a reverse transcription polymerase chain reaction (RT-PCR) alkaline gel demonstrating the BoxB RNA element-dependent skipping of DMD exon 2 in an immortalized fibroMyoD cell line transfected with AAV vectors encoding U7 snRNA constructs including asRNA complementary to DMD exon 2 and including a BoxB RNA element. Thus, a highly efficient AAV-mediated U7 snRNA, including a BoxB RNA element and an asRNA complementary to the DMD exon 2 ESE, was designed to skip DMD exon 2.

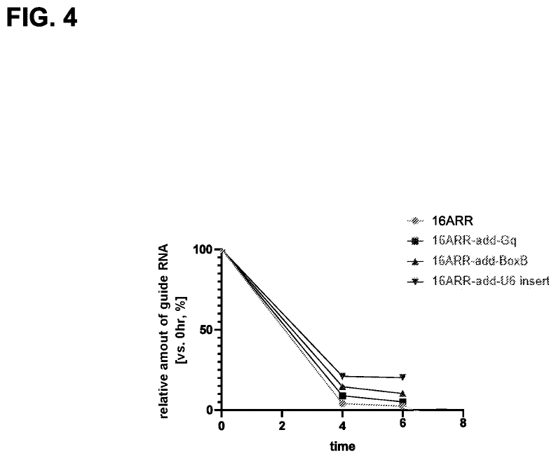

Without being limited by mechanism, the BoxB RNA element-containing compositions described herein may mediate more efficacious exon skipping than constructs without a BoxB RNA element by enhancing the expression of small antisense polynucleotides (FIG. 4; e.g., asRNA from 10 to 100 nucleotides in length having complementarity sufficient to hybridize to a region within a protein-encoding mRNA transcript e.g., dystrophin). In particular, we observed that a 3-4× greater level of expression of asRNA was observed in host cells transduced with rAAV having a BoxB RNA element, as compared to rAAV without a BoxB RNA element.

### Example 3. Induced Alternative Splicing by asRNA of DMD Exon 2

DMD is a severe, progressive, neuromuscular disorder caused by mutations in the DMD gene that result in C-terminally truncated, non-functional dystrophin proteins. The mutations that give rise to DMD are heterogeneous in nature and typically include at least one of the following: a point mutation that produces a premature termination codon, a duplication, or a deletion. Alternative splicing, for instance, by way of exon skipping, is a promising therapeutic approach for the treatment of DMD since internally truncated dystrophins can be partly functional. This is exemplified by the less severe phenotype of Becker muscular dystrophy (BMD) patients, who carry mutations in the same gene, wherein the mutations do not alter the reading frame of protein translation.

The wild-type DMD gene consists of 79 exons and nearly all patients have unique mutations. The alternative splicing approach is mutation specific because different mutations require skipping different exons to restore protein function. For example, DMD patients may have a duplication of exon 2 of the endogenous DMD gene. Thus, skipping DMD exon 2 is applicable to treatment of DMD patients.

This example describes the process for designing and administering a transgene that directs for targeted exon skipping of exon 2 resulting in the translation of a functional truncated isoform of dystrophin.

In this example, exon skipping using asRNA may be achieved by introducing into a cell a transgene including or encoding an asRNA. The efficacy of exon skipping may be further enhanced by inclusion of a BoxB RNA element into the transgene encoding an asRNA. In some embodiments, the asRNA includes a portion of from 25 to 40 (e.g., 25 to 40 (e.g., 25 to 40, 26 to 40, 27 to 40, 28 to 40, 29 to 40, 30 to 40, 31 to 40, 32 to 40, 33 to 40, 34 to 40, 35 to 40, 36 to 40, 37 to 40, 38 to 40, or 39 to 40) nucleotides in length having complementarity sufficient to hybridize over the length of, or within, a region of exon 2 of a human dystrophin RNA transcript, wherein the region begins at residue 17 of SEQ ID NO: 1 and ends at residue 46 of SEQ ID NO: 1. In some embodiments, the asRNA has a nucleic acid sequence that is at least 70% (e.g., 70% 71%, 72%, 73%, 74%, 75%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100%) complementary to the region beginning at residue 17 of SEQ ID NO: 1 and ending at residue 46 of SEQ ID NO: 1. In some embodiments, the asRNA includes from 10 to 30 (e.g., 10, 11, 12, 13, 14, 15, 16, 17, 18, 19, 20, 21, 22, 23, 24, 25, 26, 27, 28, 29, or 30) contiguous nucleotides that are fully complementary to a contiguous polynucleotide segment of equal length within the region beginning at residue 17 of SEQ ID NO: 1 and ending at residue 46 of SEQ ID NO: 1. In some embodiments, the asRNA includes 9 or fewer (e.g., 9, 8, 7, 6, 5, 4, 3, 2, 1, or 0) nucleotide mismatches relative to the region beginning at residue 17 of SEQ ID NO: 1 and ending at residue 46 of SEQ ID NO: 1. In some embodiments, the asRNA has a nucleic acid sequence that is at least 85% (e.g., 85%, 86%, 87%, 88%, 89%, 90%, 91%, 92%, 93%, 94%, 95%, 96%, 97%, 98%, 99%, 99.9%, or 100%) identical to the nucleic acid sequence of SEQ ID NO: 2. In some embodiments, the BoxB RNA element has a nucleic acid sequence that is at least 85% (e.g., 85%, 86%, 87%, 88%, 89%, 90%, 91%, 92%, 93%, 94%, 95%, 96%, 97%, 98%, 99%, 99.9%, or 100%) identical to the nucleic acid sequence of SEQ ID NO: 3.

A transgene encoding this BoxB RNA element and asRNA may be operably linked to a promoter and/or an enhancer, such as a U7 promoter and a muscle creatine kinase (MCK) enhancer, respectively, and incorporated into a viral vector, such as a recombinant single stranded (ss) or self-complementary (sc) AAV vector. The vector including the transgene may be delivered to a cell (e.g., the cell of a patient) harboring a duplication in exon 2 of the endogenous DMD gene (dup2). Following delivery of the vector, the DMD dup2 myoblasts can be induced to differentiate and samples of DMD mRNA and dystrophin protein collected after 7 days. Skipping of exon 2 can be evaluated by, e.g., RT-PCR analysis of the DMD mRNA and/or western blot of the dystrophin protein. Production of a dystrophin protein including an internal truncation is expected, thereby restoring partial function of the protein.

### Example 4. Treatment of a Muscular Dystrophy by Induced Alternative Splicing by asRNA of DMD Exon 2

Using the compositions and methods of the disclosure, a patient (e.g., a pre-ambulant or an ambulant pediatric patient from about 6 months of age to about 14 years of age) having DMD and having a duplication in exon 2 of an endogenous DMD gene may be administered a pseudotyped AAV2/8 vector including or encoding a transgene including one or more U7 snRNA expression cassettes (e.g., including a BoxB RNA element, an asRNA targeting DMD exon 2, a U7 snRNA, a Sm binding protein (U7 Sm OPT) sequence, a U7 stem loop, and a U7 downstream element), operatively linked to a U7 promoter and an MCK enhancer; a stuffer, and flanking rAAV2 inverted terminal repeat (ITR) sequences (e.g., SEQ ID NO 10).

Upon administering the viral vector including said BoxB RNA element and asRNA to the patient, the patient may exhibit increased expression of functional dystrophin protein and/or induced exon 2 skipping.

### Example 5. Induced Alternative Splicing by asRNA of DMD Exon 2 to Restore the Downstream Reading Frame in a Mutant DMD Gene

This example describes the process for designing and administering a transgene that directs for targeted exon skipping of exon 2, for example for the treatment of DMD patients may have a mutation within or affecting exon 1, 2, 3, or 4 of the DMD gene (e.g., a 5′ mutation of the DMD gene). The skipping of exon 2 may disrupt the reading frame and result in a premature stop codon, thereby activating a glucocorticoid-inducible IRES in exon 5 of the DMD gene resulting in the restoration of a 5′ mutation of the DMD gene of a 5′ mutation of the DMD gene by the translation of a functional truncated isoform of dystrophin e.g., see US 20170218366 A1.

In this example, exon skipping using asRNA may be achieved by introducing into a cell an antisense polynucleotide. The efficacy of alternative splicing may be enhanced by the further inclusion of a BoxB RNA element. In some embodiments, the asRNA includes a portion of from 25 to 40 (e.g., 25 to 40 (e.g., 25 to 40, 26 to 40, 27 to 40, 28 to 40, 29 to 40, 30 to 40, 31 to 40, 32 to 40, 33 to 40, 34 to 40, 35 to 40, 36 to 40, 37 to 40, 38 to 40, or 39 to 40) nucleotides in length having complementarity sufficient to hybridize over the length of, or within, a region of exon 2 of a human dystrophin RNA transcript, wherein the region begins at residue 17 of SEQ ID NO: 1 and ends at residue 46 of SEQ ID NO: 1. In some embodiments, the asRNA has a nucleic acid sequence that is at least 70% (e.g., 70% 71%, 72%, 73%, 74%, 75%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100%) complementary to the region beginning at residue 17 of SEQ ID NO: 1 and ending at residue 46 of SEQ ID NO: 1. In some embodiments, the asRNA includes from 10 to 30 (e.g., 10, 11, 12, 13, 14, 15, 16, 17, 18, 19, 20, 21, 22, 23, 24, 25, 26, 27, 28, 29, or 30) contiguous nucleotides that are fully complementary to a contiguous polynucleotide segment of equal length within the region beginning at residue 17 of SEQ ID NO: 1 and ending at residue 46 of SEQ ID NO: 1. In some embodiments, the asRNA includes 9 or fewer (e.g., 9, 8, 7, 6, 5, 4, 3, 2, 1, or 0) nucleotide mismatches relative to the region beginning at residue 17 of SEQ ID NO: 1 and ending at residue 46 of SEQ ID NO: 1. In some embodiments, the asRNA has a nucleic acid sequence that is at least 85% (e.g., 85%, 86%, 87%, 88%, 89%, 90%, 91%, 92%, 93%, 94%, 95%, 96%, 97%, 98%, 99%, 99.9%, or 100%) identical to the nucleic acid sequence of SEQ ID NO: 2. In some embodiments, the BoxB RNA element has a nucleic acid sequence that is at least 85% (e.g., 85%, 86%, 87%, 88%, 89%, 90%, 91%, 92%, 93%, 94%, 95%, 96%, 97%, 98%, 99%, 99.9%, or 100%) identical to the nucleic acid sequence of SEQ ID NO: 3.

A transgene encoding this asRNA may be operably linked to a promoter and/or an enhancer, such as a U7 promoter or a MCK enhancer, respectively, and incorporated into a viral vector, such as a recombinant ss or sc AAV vector. The vector including the transgene may be delivered to a cell (e.g., the cell of a patient) harboring a frameshift mutation in any one of exons 1-4 of an endogenous DMD gene (e.g., A5′). Following delivery of the vector, the A5′ DMD myoblasts can be induced to differentiate and samples of DMD mRNA and dystrophin protein collected after 7 days. Skipping of exon 2 can be evaluated by, e.g., RT-PCR analysis of the DMD mRNA and/or western blot of the dystrophin protein. Production of a dystrophin protein including an internal truncation is expected, thereby restoring the downstream reading frame and partial function of the protein.

### Example 6. Treatment of a Muscular Dystrophy by Induced Alternative Splicing by asRNA of DMD Exon 2 to Restore the Downstream Reading Frame in a Mutant DMD Gene

Using the compositions and methods of the disclosure, a patient (e.g., a pre-ambulant or an ambulant pediatric patient from about 6 months of age to about 14 years of age) having DMD and having a frameshift mutation in any one of exons 1-4 of an endogenous DMD gene may be administered a pseudotyped AAV2/8 vector including or encoding a nucleic acid sequence of one or more U7 snRNA expression cassette (e.g., including a BoxB RNA element, an asRNA targeting DMD exon 2, a U7 snRNA, a U7 Sm OPT sequence, a U7 stem loop, and a U7 downstream element), operatively linked to a U7 promoter and an MCK enhancer; a stuffer, and flanking rAAV2 ITR sequences (e.g., SEQ ID NO 10).

Upon administering the viral vector including said BoxB RNA element and asRNA to the patient, the patient may exhibit increased expression of functional dystrophin protein and/or activation of an internal ribosomal entry site within exon 5 of an endogenous DMD gene.

### OTHER EMBODIMENTS

All publications, patents, and patent applications mentioned in this specification are incorporated herein by reference to the same extent as if each independent publication or patent application was specifically and individually indicated to be incorporated by reference.

While the presently disclosed subject matter has been described in connection with specific embodiments thereof, it will be understood that it is capable of further modifications and this application is intended to cover any variations, uses, or adaptations of the presently disclosed subject matter following, in general, the principles of the presently disclosed subject matter and including such departures from the presently disclosed subject matter that come within known or customary practice within the art to which the presently disclosed subject matter pertains and may be applied to the essential features hereinbefore set forth, and follows in the scope of the claims.

Other embodiments are within the claims.

1-114. (canceled)

115. A transgene encoding a ribonucleic acid (RNA) molecule, wherein the RNA molecule comprises:
(i) a BoxB RNA element; and
(ii) an antisense polynucleotide of from 10 to 100 nucleotides in length having complementarity sufficient to hybridize to a region within a protein-encoding mRNA transcript.

116. The transgene of claim 115, wherein the BoxB RNA element has a nucleic acid sequence that is at least 85% identical to the nucleic acid sequence of SEQ ID NO: 3.

117. The transgene of claim 115, wherein the mRNA transcript is a human dystrophin mRNA transcript, a human fukutin mRNA transcript, a human gamma-sarcoglycan mRNA transcript, a human dysferlin mRNA transcript, a human myotonic dystrophy protein kinase mRNA transcript, a human laminin subunit alpha 2 mRNA transcript, a human usherin mRNA transcript, a human collagen alpha-1(VII) chain mRNA transcript, or a human activin A receptor type 1 mRNA transcript.

118. The transgene of claim 115, wherein the antisense polynucleotide comprises a portion of from 30 to 40 nucleotides in length having complementarity sufficient to hybridize over the length of a region of exon 2 of a human dystrophin mRNA transcript, optionally wherein the antisense polynucleotide is from 30 to 40 nucleotides in length and has complementarity sufficient to hybridize over the length of a region of exon 2 of a human dystrophin mRNA transcript, optionally wherein the antisense polynucleotide is from 30 to 39 nucleotides in length, from 30 to 38 nucleotides in length, from 30 to 37 nucleotides in length, from 30 to 36 nucleotides in length, or 30 nucleotides in length.

119. The transgene of claim 115, wherein the antisense polynucleotide has a nucleic acid sequence that is at least 70% complementary to the region beginning at residue 17 of SEQ ID NO: 1 and ending at residue 46 of SEQ ID NO: 1.

120. The transgene of claim 115, wherein the antisense polynucleotide has a nucleic acid sequence that is at least 85% identical to the nucleic acid sequence of SEQ ID NO: 2.

121. The transgene of claim 115, wherein the RNA molecule further comprises a U7 small nuclear RNA (snRNA).

122. The transgene of claim 121, wherein the U7 snRNA has a nucleic acid sequence that is at least 85% identical to the nucleic acid sequence of SEQ ID NO: 8.

123. A composition comprising the transgene or RNA molecule of claim 115, wherein the composition is a liposome, vesicle, synthetic vesicle, exosome, synthetic exosome, dendrimer, or nanoparticle.

124. A viral vector comprising the transgene of claim 115.

125. A pharmaceutical composition comprising the viral vector of claim 124 and a pharmaceutically acceptable carrier, diluent, or excipient.

126. A method of treating Duchenne muscular dystrophy in a human patient diagnosed as having a duplication in exon 2 of an endogenous DMD gene or a frameshift mutation in any one of exons 1-4 of an endogenous DMD gene, the method comprising administering to the patient a therapeutically effective amount of the pharmaceutical composition of claim 125.

127. A kit comprising (i) the pharmaceutical composition of claim 125, and (ii) a package insert, wherein the package insert instructs a user of the kit to administer the composition to a human patient diagnosed as having Duchenne muscular dystrophy.
